(19) United States
(12) Patent Application Publication
(10) Pub. No.: US 2025/0195281 A1
(43) Pub. Date: Jun. 19, 2025

(54) FLUID MANAGEMENT LAYER FOR AN ABSORBENT ARTICLE

(71) Applicant: The Procter & Gamble Company, Cincinnati, OH (US)

(72) Inventors: Gerard Alain VIENS, Wyoming, OH (US); Olivia Maeve NEWMAN, Cincinnati, OH (US); Matteo GHIOLDI, Frankfurt (DE)

(21) Appl. No.: 18/978,106

(22) Filed: Dec. 12, 2024

Related U.S. Application Data
(60) Provisional application No. 63/610,672, filed on Dec. 15, 2023.

(51) Int. Cl. A61F 13/15; A61F 13/472; A61F 13/53; A61F 13/539; A61L 15/24; A61L 15/26; A61L 15/28; A61L 15/42

(52) U.S. Cl. CPC A61F 13/15203; A61F 13/539; A61L 15/24; A61L 15/26; A61L 15/28; A61L 15/425; A61F 2013/15373; A61F 2013/15406; A61F 13/472; A61F 2013/53016; A61F 2013/530182; A61F 2013/53024; A61F 2013/530343; A61F 2013/530379; A61F 2013/530416; A61F 2013/53908

(57) ABSTRACT

A fluid management layer including a nonwoven having a basis weight of from about 40 gsm to about 85 gsm. The nonwoven includes from about 15 to about 35 weight percent of cellulosic fibers, from about 20 to about 40 weight percent of bonding fibers, and from about 40 to about 60 weight percent of divider fibers. The fluid management layer has a caliper factor of from about 0.26 to about 0.40, and each of the cellulosic fibers and the bonding fibers have a decitex no greater than about 2.

### CROSS REFERENCE TO RELATED APPLICATIONS

This application claims the benefit, under 35 U.S.C. § 119 (e), to U.S. Provisional Application No. 63/610,672 filed Dec. 15, 2023, the entire disclosure of which is fully incorporated by reference herein.

### FIELD OF THE INVENTION

The present disclosure generally relates to fluid management layer for a disposable absorbent article, in particular, a fluid management layer that is a needle punched nonwoven layer having improved performance characteristics.

### BACKGROUND OF THE INVENTION

Disposable absorbent articles such as feminine hygiene products, taped diapers, pant-type diapers, and incontinence products are designed to absorb fluids from the wearer's body. Users of such disposable absorbent articles have several concerns. For example, leakage from products like catamenial pads, diapers, sanitary napkins, and incontinence pads is a significant concern. Additionally, comfort and the feel of the product against the wearer's body is also a concern. To provide better comfort, current disposable absorbent articles are typically provided with a topsheet that is flexible, soft feeling, and non-irritating to the wearer's skin. The topsheet does not itself hold the discharged fluid. Instead, the topsheet is fluid-permeable to allow the fluids to flow into an absorbent core.

Regarding comfort, some consumers may desire a product that has sufficient thickness and stiffness to provide the desirable amount of protection while also being flexible. Lofty materials may be utilized to provide a thick cushiony feeling article. However, in use these lofty materials can experience various compressive loads. Recovery from these compressive loads is paramount in maintaining the cushiony feeling of the article. Exacerbating this issue is the fact that the characteristics of the materials of the absorbent article change once fluid is introduced into the article. Hence, an article that may meet a consumer's requisite criteria before use may no longer be comfortable, flexible, or have the desired stiffness to the user after a given amount of fluid has been absorbed by the absorbent article.

As such there is a need to create fluid management materials that have sufficient caliper and consumer desired recovery properties for use in absorbent articles while still delivering sufficient capillary action to facilitate fluid moving from the topsheet of the product to the absorbent core.

### SUMMARY OF THE INVENTION

In some embodiments, a fluid management layer includes: a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers. The fluid management layer has a caliper factor of from about 0.26 to about 0.40, and wherein the cellulosic fibers and the bonding fibers have a decitex no greater than about 2.

In some embodiments, a disposable absorbent article includes a topsheet, a backsheet, an absorbent core disposed between the topsheet and the backsheet, and a fluid management disposed between the topsheet and the absorbent core. The fluid management layer includes a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers. The fluid management layer has a caliper factor of from about 0.26 to about 0.40, and the cellulosic fibers and the polymeric fibers have a decitex less than about 2.

In some embodiments, a fluid management layer includes: a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers. The cellulosic fibers, the bonding fibers, and the divider fibers have a decitex of less than about 2.

### BRIEF DESCRIPTION OF THE DRAWINGS

FIG. 1 is a schematic representation of a disposable absorbent article constructed in accordance with the present disclosure;

FIG. 1A is a schematic representation of a disposable absorbent article constructed in accordance with the present disclosure;

FIG. 2 is a schematic representation of a disposable absorbent article constructed in accordance with the present disclosure;

FIG. 3 is fluid management layer top view (no cross lapping for comparison);

FIG. 4 is a fluid management layer top view with needlepunch;

FIG. 5 is a fluid management layer top view with needlepunch cross section with vertical needle bundle;

FIG. 6 is a fluid management layer top view with areas of capillary boosting points;

FIGS. 7-10 include equipment for Acquisition Time and Rewet Method;

FIGS. 11-15 include views of equipment and graphs for the Bunch Compression & Bending Length test;

FIGS. 16a-18 include views of equipment for the Permeability Measurement Method; and

FIGS. 19-20 include views of a fiber layers and views to aid in determining the percent bonded volume using the % Bonded Volume Measurement via Micro-CT Method.

### DETAILED DESCRIPTION OF THE INVENTION

### Definitions

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene products, including feminine hygiene pads (also known as, for example, “sanitary napkins”, “menstrual pads”, “panty liners”, etc.).

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydro-enhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of the receiving layer described herein. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e., the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

The term “lateral” as used herein with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

The term “longitudinal” as used herein with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

The term “x-y plane,” with reference to an absorbent article, such as a feminine hygiene pad, or component thereof, when laid out flat on a horizontal surface, means any horizontal plane occupied by the horizontal surface or any layer of the article or component.

The term “z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a receiving layer has a wearer-facing surface that lies closer to the wearer than the opposite, outward-facing surface of the receiving layer.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a receiving layer has an outward-facing surface that lies farther from the wearer than the opposite, wearer-facing surface of the receiving layer.

“Machine Direction” or “MD” as used herein with respect to an absorbent article such as a feminine hygiene pad or component thereof, refers to a direction parallel to the flow of the article or component through processing/manufacturing equipment.

“Cross Machine Direction” or “CD” as used herein with respect to an absorbent article such as a feminine hygiene pad or component thereof, refers to a direction perpendicular/orthogonal to the machine direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### Absorbent Article—Feminine Hygiene Pad

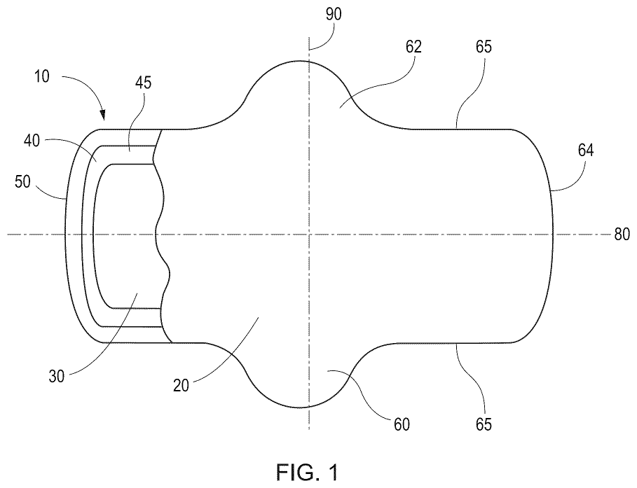

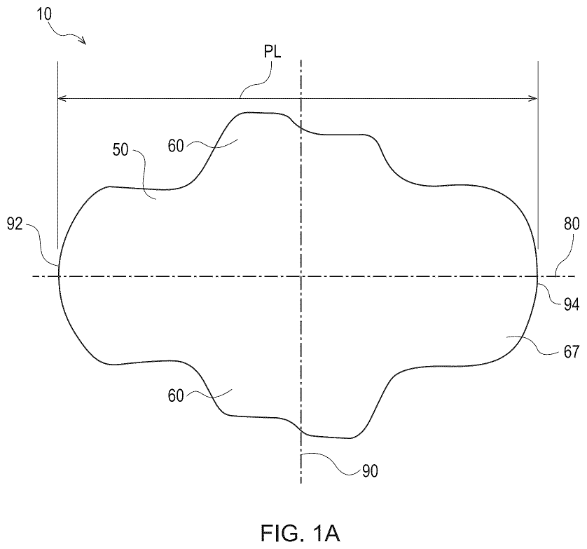

Referring to FIG. 1, an absorbent article as contemplated herein, such as a feminine hygiene pad 10, will include a wearer-facing surface and an opposing outward-facing surface. A liquid permeable topsheet 20 may form at least a portion of the wearer-facing surface and a liquid impermeable backsheet may form at least a portion of the outward-facing surface. An absorbent core including an absorbent structure 40 is disposed between the topsheet and the backsheet, and a fluid management layer 30 may be included and disposed between the absorbent structure 40 and the topsheet 20. (A fluid management layer as described herein is sometimes known in the art as an “acquisition/distribution layer”, “distribution layer”, or “secondary topsheet”, whose purpose is to dissipate energy from a fluid gush to the extent needed, provide a temporary volume of space for discharged fluid to occupy during the time required for an underlying absorbent structure to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants.

The topsheet 20 and the backsheet 50 may be joined together to form and define an outer periphery 65 of the pad 10. The absorbent structure 40, including an absorbent core 45, and the fluid management layer 30 may each be sized to have outer perimeters disposed laterally and longitudinally inboard of the outer periphery 65. The absorbent structure 40 and the fluid management layer 30 may be dimensioned and shaped substantially similarly or identically to each other in the x-y directions, or they may have respective differing x-y dimensions and/or shapes.

One or both may be manufactured to have a rectangular shape as suggested in FIG. 1, or one or both may be manufactured to have any other suitable shape, such as an oval shape, stadium shape, rounded rectangle shape, hourglass shape, peanut shape, etc. Shapes having concave profiles along the longitudinal edges may in some examples provide for enhanced comfort and/or conformity with the wearer's body.

The topsheet 20 may be joined to the backsheet 50 by any suitable attachment mechanism. The topsheet 20 and the backsheet 50 may be joined directly to each other in the article periphery 65 and may be indirectly joined together by directly joining them to the absorbent structure 40, the fluid management layer 30, and/or additional layers disposed between the topsheet 20 and the backsheet 50. This indirect or direct joining may be accomplished by any suitable attachment mechanism known in the art. Non-limiting examples of attachment mechanisms may include e.g., fusion bonds, ultrasonic bonds, pressure bonds, adhesive bonds, or any suitable combinations thereof. The absorbent article 10 may also comprise wings 60 extending outwardly with respect to a longitudinal axis 80 of the absorbent article 10. As illustrated in FIG. 1A, the wings may be asymmetric, such as disclosed, for example, in U.S. patent publication numbers 2022/0409449, 2021/0307977, 2018/0325750, and 2018/0325751, which are all incorporated herein by reference. The wings may be asymmetric about at least one of the longitudinal axis 80 and the lateral axis 90. The pad may have a pad length PL taken parallel to longitudinal axis from the first lateral edge 92 to the second lateral edge 94. The wings 60 may be positioned in the central region of the absorbent article, such as illustrated in FIG. 1A.

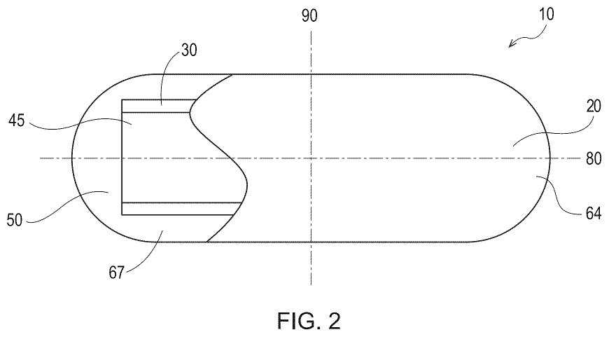

In some embodiments, referring to FIG. 2, the disposable absorbent article 10 having a topsheet 20, a backsheet 50, an absorbent core 45, disposed between the topsheet and the backsheet, and a fluid management layer 30 disposed between the topsheet and the absorbent core. As illustrated in FIG. 2, the absorbent article 10 may not have a wing. Further, the absorbent core 45 and the fluid management layer 30 may be sized such that a portion of the absorbent core 45 extends beyond one or more sides of the fluid management layer 30 and/or a portion of the fluid management layer 30 extends beyond one or more side edges of the absorbent core 45. The topsheet 20 may form the wearer-facing surface of the pad, such that the topsheet is the first layer to receive any bodily exudates. The fluid management layer 30 may disposed adjacent the topsheet 20 and be positioned between the topsheet 20 and the absorbent core 45. The fluid management layer 30 aid in wicking the bodily fluid away from the topsheet quickly and transferring the fluid to the absorbent core. The topsheet and the fluid management layer work together to quickly absorb and transfer fluid. As previously discussed, the absorbent core 45 holds to the bodily fluid and the backsheet 50 protects the wearer's undergarments by not allowing fluid to pass through the backsheet layer. The backsheet provides the garment facing layer of the absorbent article.

As described, there are two layers, the topsheet layer and the fluid management, layer that work together to absorb and wick fluid. Traditionally, these two layers are manufactured separately and the topsheet web and the fluid management web are stored on rolls that are to be used on a converting manufacturing line. The rolls are supplied to the converting manufacturing line where the two separate materials are unwound, processed, and assembled into an absorbent article. The topsheet layer and the fluid management layer are typically bonded to one another by adhesive or other bonding at specific bond sites.

The fluid management layer 30 is critical for the topsheet to remain dry-feeling against the wearer's skin and to manage fluid so that it does not leak and can be absorbed and stored in the absorbent core. To optimize these properties, the fluid management layer 30 of the present disclosure comprises a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers. The fluid management layer can have a caliper factor (mm per 10 gsm) of from about 0.26 to about 0.40, and at least two of the cellulosic fibers, the bonding fibers, and the divider fibers can have a decitex below about 2. The absorbent article can have a Z-compression compression energy of from about 2.6 N·mm to about 4.0 N·mm, a 3 point MD bend dry bending stiffness of from about 15 N·mm\hat{ }2 and about 55 N·mm\hat{ }2, and a wet bunch compression % recovery of greater than about 40% or from about 40% to about 70%.

### Topsheet

Generally, it is desirable that the topsheet 20 be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of a lotion composition onto an external or internal portion of a wearer's skin. The topsheet may comprise a nonwoven material.

Nonwoven fibrous topsheets 20 may be produced by any known procedure for making nonwoven webs, nonlimiting examples of which include spunbond processes, carding, wet-laid, air-laid, meltblowing processes, needle-punching, mechanical entangling, 8opshee-mechanical entangling, and hydroentangling.

Nonwoven materials suitable for use as a topsheet may include one strata of fibers or may be a laminate of multiple nonwoven strata, which may comprise the same or different compositions (e.g., spunbond-meltblown laminate, spunbond-meltblown-spunbond laminate). In one specific example, the topsheet is a carded, air-through bonded nonwoven.

Some currently known topsheets for feminine hygiene pads include an apertured film, such as a hydroformed film or vacuum-formed film, alone or in combination with an adjacently-disposed nonwoven web material. The film may help to prevent liquids from resurfacing and contacting the wearer. The inventors have found, however, that a topsheet having the features described herein, particularly in combination with the fluid management layer described herein, can effectively prevent rewet to a comparable degree or better, than pads having topsheets comprising film across a predominant portion of topsheet x-y surface area. Without being bound by theory, it is believed that the careful selection of the fiber types in each of the strata in the fluid management layer, and the linear densities of the fiber types, can result in a desired combination of suitably fast acquisition, and low rewet, overcoming the typical tradeoff in these conflicting objectives associated with prior nonwoven topsheets. The improved performance is evident from the new combination of the unique nonwoven topsheet with a fluid management layer of the present disclosure.

The8opsheett nonwoven may be manufactured to a basis weight of at least about 10 gsm up to about 60 gsm, or from about 15 gsm to about 50 gsm, or from about 20 gsm to about 45 gsm, specifically reciting all values within these ranges and any ranges created thereby. In some examples, a nonwoven topsheet contemplated herein may be manufactured to have a basis weight of about 15 gsm to 60 gsm, or from about 18 gsm to 40 gsm, or, alternatively, from about 20 gsm to 30 gsm, specifically reciting all values within these ranges and any ranges created thereby. Suitable topsheet nonwoven may be manufactured to have a basis weight of about 18 gsm to 40 gsm, alternatively from about 20 gsm to 30 gsm, alternatively from about 22 gsm to 26 gsm, specifically reciting all values within these ranges and any ranges created thereby. The range of desirable basis weight is influenced, at the lower end of the range, by the need for a level of web tensile strength required for processing, and by consumer preferences for a level of opacity and substantiality of loft, feel, and appearance. The range of desirable basis weight is influenced, at the upper end of the range, by the need for suitable rapid fluid acquisition and passage of fluid through the topsheet, and material cost concerns.

### Fiber Composition

Nonlimiting examples of woven and nonwoven materials suitable for use as the topsheet include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers. Synthetic fibers may include monocomponent fibers, bicomponent fibers or multicomponent fibers. (Herein, bi- or multicomponent fibers are fibers having cross sections divided into distinctly identifiable component sections each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven topsheets contemplated herein may include fibers having myriad combinations of constituent chemistries. For example, fibers may be spun from polymeric materials, such as polyethylene (PE) and/or polyethylene terephthalate (PET). Fibers may be spun in the form of bi-component fibers. In some examples, bi-component fibers may have a core component of a first polymer (for example, PET) in combination with another polymer as a sheath component, in a sheath-core bicomponent configuration. In more particular examples, PE may form the sheath component in combination with a PET core component. Fibers that include a PET component may be selected to help provide bulk and resilience and a resulting cushiony feel to the nonwoven web. Additionally, fibers that include a PET component, having resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component where sheath-core bi-component fibers are included. Additionally, without wishing to be bound by theory, it is believed that the use of polyethylene terephthalate as a core can help impart resilience to the topsheet.

Polyethylene, as a polymer component from which fibers may be spun, has a relatively lower melting temperature, and exhibits a relatively slick/silky surface feel as compared with other potentially useful polymers. PET has a relatively higher melting temperature and exhibits relatively greater stiffness and resiliency. Accordingly, in some examples topsheet nonwoven fibers that are of a sheath-core bicomponent configuration may be desired, in which the sheath component is predominantly polyethylene, and the core component is predominantly PET. The polyethylene is useful for imparting the fibers and thus the topsheet with a silky feel, and for enabling inter-fiber bonding via heat treatment that cause sheaths of adjacent/contacting fibers to melt and fuse at the lower melting temperature of the polyethylene, while the PET is useful for imparting resilience, and does not melt in the heat treatment process. The inventors have found that a suitable weight ratio in such PE/PET sheath-core bicomponent fibers may be about 40:60 to about 60:40.

Depending upon the chemical composition thereof, surfaces of fibers will be, inherently, either hydrophilic or hydrophobic, to varying extents. For example, surfaces of fibers spun or otherwise formed from some types of polymers such as polyethylene and polypropylene will be, inherently, hydrophobic. In contrast, surfaces of other types of fibers, such as rayon fibers, will be inherently hydrophilic. Surfaces of natural fibers may be inherently hydrophilic or hydrophobic, but this may depend upon the processing the fibers have undergone. For example, cotton fibers as harvested bear coatings of natural oils and/or waxes and as such their surfaces are hydrophobic. After they have undergone processes including scouring and bleaching, however, the oils and/or waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, e.g., carding processes. These surface finishing agents or processing aids may be formulated to be either hydrophobic or hydrophilic, and to be substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes.

Noting that spun synthetic staple fibers may be obtained with either inherently hydrophobic or hydrophilic surfaces, or obtained with surface finishes that render their surfaces hydrophilic or hydrophobic at the purchaser's option, it may be desirable to choose fibers with surfaces that are either hydrophilic (“hydrophilic fibers”) or hydrophobic (“hydrophobic fibers”), or choose a blend of fibers of both types. In some examples it may be preferable that the fiber constituents of the topsheet be, by weight, predominantly, substantially, or entirely hydrophobic, or rendered hydrophobic via fiber surface finish. A topsheet formed of a nonwoven web with predominately hydrophobic fiber constituents will be resistive to rewetting. On the other hand, if the sizes of the pores or inter-fiber voids within the fibrous structure of such nonwoven web are not sufficiently large, the topsheet may resist the passage of fluid from the wearing facing surface through to the absorbent core components of the article therebeneath, i.e., will not readily/rapidly acquire fluid, unless other features are included in combination, as described herein.

In other examples, fibers constituting portions, a majority (by surface area), or all, of the section of web material from which of the topsheet is formed, may be a blend of both hydrophobic fibers and hydrophilic fibers. In such examples, the hydrophilic fibers can serve to help wick fluid from the wearer-facing surface of the topsheet down to the absorbent core components beneath, while the hydrophobic fibers can serve to help the topsheet resist rewetting. The inventors have discovered that a successful balance may be struck for such examples. Accordingly, in some examples the topsheet nonwoven may include a mix of hydrophobic and hydrophilic fibers. For example, the nonwoven may include at least about 40 percent, alternatively at least about 50 percent, or alternatively at least about 60 percent hydrophilic fibers by weight of the fibers, specifically including all values within these ranges and any ranges created thereby. In more particular examples, the nonwoven topsheet may comprise about 40 percent to 70 percent, alternatively about 45 percent to 68 percent, or alternatively from about 50 percent to 65 percent, by weight, hydrophilic fibers, specifically reciting all values within these ranges and any ranges created thereby. The topsheet nonwoven may include a blend of hydrophilic fibers and hydrophobic fibers in a weight ratio of hydrophilic fibers to hydrophobic fibers of 30:70 to 70:30, alternatively 35:65 to 65:35, and alternatively 40:60 to 60:40. As noted above, the hydrophilicity of the hydrophilic fibers may be effected by application of a surface treatment composition.

Without wishing to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

Fibers are typically manufactured, selected and purchased by linear density specification, such expressed as denier or decitex. For fibers of a given polymer constitution, linear density correlates with fiber size/diameter. In some examples, the fibers constituting the topsheet may be selected to have an average linear density of about 1.0 to 3.0 denier, alternatively about 1.5 to 2.5 denier, and alternatively about 1.8 to 2.2 denier, and all combinations of subranges within these ranges are contemplated herein. Fibers with varying linear densities within the ranges set forth above may be selected and included as well.

In other examples, the fibers constituting the topsheet may be selected to have an average linear density of about 3.0 to 5.0 denier, alternatively about 3.5 to 4.5 denier, and alternatively about 3.8 to 4.2 denier, and all combinations of subranges within these ranges are contemplated herein. It has been learned that fibers selected within these ranges, in combination with other features disclosed herein, may be deemed to constitute a topsheet material of acceptable softness to many consumers, as well as to provide other advantages over smaller fibers.

One advantage is that the relatively larger fibers generally provide a nonwoven web material with relatively larger inter-fiber/intra-web spaces or voids therewithin, thereby providing larger passageways through which fluid may more rapidly travel through the nonwoven from the wearer-facing side through to the outward-facing side (and thus to absorbent components below the topsheet). Additionally, although relatively larger fibers of a given composition are stiffer than smaller fibers of similar composition, which may somewhat compromise surface “softness” attributes, the greater fiber stiffness can also enhance a feeling of greater resiliency, springy or cushiony feel to the topsheet nonwoven.

The topsheet may include staple fibers having a length of at least about 30 mm, 40 mm, or 50 mm, up to about 55 mm, or about 30 to 55 mm, or about 35 to 52 mm, reciting for said range every 1 mm increment therein. In particular example, staple fibers may have a length of about 38 mm.

The absorbent article may include an anti-stick agent applied, to at least a portion of the wearer-facing surface of the topsheet, wherein the anti-stick agent includes a polypropylene glycol material. It is believed that an applied anti-stick agent as described herein serves functions that include reducing adherence of menstrual fluid to the user/wearer's skin, and facilitation of migration of menstrual fluid from the wearer-facing surface of the topsheet, down therethrough to the fluid management and/or absorbent structure layers beneath. Serving these functions can enhance user/wearer perceptions of cleanliness of her skin and of the topsheet, especially after repeated discharges of menstrual fluid. Examples of a suitable anti-stick agents and/or surfactants useful therein are disclosed in US 2009/0221978 (wherein the composition is called a “lotion”) and U.S. Pat. No. 8,178,748.

The anti-stick agent may include a polypropylene glycol (“PPG”) material. In some examples, the anti-stick agent may consist essentially of a polypropylene glycol material, including but not limited to polypropylene glycol homopolymer such as polypropylene glycol, and optionally, a carrier. In other examples, the anti-stick agent may include a polypropylene glycol material selected from the group of polypropylene glycol copolymer, polypropylene glycol surfactant, and mixtures thereof. The anti-stick agent including the polypropylene glycol material may serve to help reduce the adherence of menstrual fluid to the topsheet, and upon contact transfer of anti-stick agent to the user/wearer, reduce the adherence of fluid to her skin, thereby reducing staining on the topsheet and reducing soiling of the skin. The anti-stick agent may also help to improve continuous fluid acquisition of the absorbent article.

The anti-stick agent may be applied in any known manner, in any known pattern, and to the wearer-facing surface of the topsheet 20. For example, the anti-stick agent may be applied in a pattern of generally parallel, longitudinally- or laterally-oriented stripes or bands. To avoid compressing or displacing any portion of the topsheet nonwoven or any three-dimensional features thereof, it may be desired that the anti-stick agent be applied via spraying. The spray can be applied substantially uniformly.

The quantity of anti-stick agent applied may vary and can be adjusted for specific needs. For example, while not being bound by theory, it is believed that anti-stick agent may be applied at levels that are effective, of at least about 0.1 gsm, 0.5 gsm, 1 gsm, 2 gsm, 3 gsm, 4 gsm, 5 gsm, 10 gsm, up to about 15 gsm, or up to about 12 gsm, or up to about 10 gsm. It is believed that efficacy is not further enhanced above these upper limits, and so applications at basis weights exceeding these upper limits may be needless usage (waste) of anti-stick agent. The anti-stick agent can be applied within any subrange defined by any of the levels recited above (e.g., from about 0.1 gsm to about 15 gsm). These levels refer to the area of the topsheet surface to which the anti-stick agent is actually applied. The anti-stick agent can be applied on the majority, substantially all, or all of the surface area of the topsheet overlying the fluid management layer and/or absorbent core. This is because, as is believed, the anti-stick agent may enhance the ability of the topsheet to resist rewetting.

The anti-stick agent contemplated herein offers significant advantages over other anti-stick agents, including non-PPG derived surfactants and other surface modifying agents. The advantages may be deemed particularly useful for feminine hygiene pads. Without intending to be bound by theory, it is believed that the superior fluid handling properties of the PPG materials identified herein is a result of the way in which the PPG materials act on the solid components of menstrual fluid, as opposed to surface energy treatments which act on the water component of menses. Surface energy treatments may be less effective due to the presence of polar and dispersive components in menstrual fluid, which may inhibit the effectiveness of surface energy treatments. Because the PPG materials identified herein are typically not readily soluble in menstrual fluid, they can effectively coat surfaces without dissolving in the fluid, which provides a hydrated barrier whose electron donating dipoles repel negatively dipoled proteins, thereby rendering the menstrual fluid less apt to adhere to surfaces of the article or the wearer's skin. Less adherence of menstrual fluid to the wearer's skin and/or to the topsheet promotes better and faster fluid movement through the topsheet, and fewer, smaller and/or less visible stain patterns on used products.

The PPG materials identified herein can be applied as one component in an anti-stick agent, or can be applied neat (i.e., the anti-stick agent consists of PPG material). PPG materials, either neat and/or as part of an anti-stick agent, can be applied at varying quantity levels, depending on the fluid handling properties desired and desired treatment of the wearer's skin. PPG materials may be applied to the outer surface of the topsheet in any pattern, such as full coat, stripes or bands (oriented in the MD or CD direction), droplets, spiral patterns, and other patterns. An anti-stick agent including the PPG material may also be disposed near channels or embossed areas, when present.

The anti-stick agent contemplated herein may include a PPG material. PPG materials suitable for purposes contemplated herein include PPG homopolymer materials, PPG copolymer materials, and PPG surfactant materials, as well as mixtures thereof. The anti-stick agent may further comprise other optional ingredients. Suitable anti-stick agents include a PPG material, including but not limited to polypropylene glycol. Alternatively, the anti-stick agent comprises a PPG material selected from the group consisting of polypropylene glycol copolymer, polypropylene glycol surfactant, and mixtures thereof.

The anti-stick agents contemplated herein may include a PPG material at a level of about 0.1% to 100%, by weight of the anti-stick agent. In some examples, the anti-stick agent may include less than about 10%, alternatively from about 0.5% to 8%, and alternatively from about 1% to 5% of a PPG material, by weight of the anti-stick agent. The anti-stick agent may include at least about 50%, alternatively from about 75% to 100%, and alternatively from about 90% to 100% of a PPG material, by weight of the anti-stick agent.

Suitable PPG homopolymer materials may include those corresponding to the following formula:

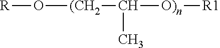

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R can be a hydrogen or methyl;
wherein R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R1 can be a hydrogen or methyl; and
wherein n is from 3 to 160, alternatively from 5 to 120, alternatively from 10 to 100, and alternatively from 20 to 80.

Optionally, the PPG homopolymer may include low level of glycerol or butanediol as part of its monomer raw material. If included, suitable ratios of glycerol or butanediol to propylene glycol may be about 1:1000 to about 1:2, alternatively from about 1:100 to about 1:5. The PPG homopolymer may have, but is not necessarily limited to, CAS Numbers 25322-69-4, 25791-96-2 and 25231-21-4.

Non-limiting examples of suitable PPG homopolymer materials include polypropylene glycol 4000 such as Pluriol P-4000 (BASF), Alkapol PPG-4000 (Alkaril Chemical) and Niax Polyol PPG 4025 (Union Carbide); polypropylene glycol 3500; polypropylene glycol 3000 such as Niax PPG 3025 (Union Carbide); polypropylene glycol 2000 such as Alkanol PPG-2000 (Alkaril Chemical) and Pluriol P-2000 (BASF), polypropylene glycol 1200 such as Alkapol PPG-1200 (Alkaril Chemical) and Glucam P-20 Humectant (Noveon); polypropylene glycol 1000 such as Niax PPG 1025 (Union Carbide); polypropylene glycol 600 such as Alkanol PPG-600 (Alkaril Chemical) and Glucam P-10 Humectant (Noveon); polypropylene glycol 400 such as Alkanol PPG-425 (Alkaril Chemical). polypropylene glycol 4000 glycerol ether such as Pluriol T-4000 (BASF); polypropylene glycol 2000 glycerol ether, polypropylene glycol 2000 butanediol ether, polypropylene glycol 1500 glycerol ether such as Pluriol T-1500 (BASF), polypropylene glycol 4000 with monomethyl ether, polypropylene glycol 2000 with dimethyl ether, polypropylene glycol 4000 with monobutyl ether, polypropylene glycol 2000 with monobutyl ether, polypropylene glycol 1200 with dibutyl ether, polypropylene glycol 4000 with bis(2-aminopropyl ether), PPG-10 methyl glucose ether and PPG-20 methyl glucose either.

Suitable PPG homopolymer materials will typically have a number average molecular weight of about 400 to 10,000, alternatively about 600 to about 6,000, and alternatively from about 1,200 to about 4,800.

Suitable PPG copolymer materials include those in which the polypropylene glycol segments are present as an internal block component and/or as a terminal component, of the copolymer structure. The following formulae illustrate the internal block components and terminal block components:

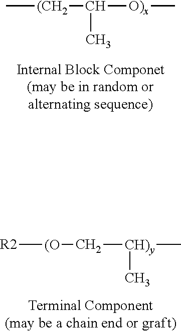

wherein x is 2 to 120, alternatively 2 to 80, and alternatively 3 to 60; y is 2 to 100, alternatively 2 to 50, and alternatively 3 to 30; R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate, and wherein R2 is hydrogen, methyl, ethyl, isopropyl or isobutyl.

Polymers suitable to form propoxylated copolymers with PPG for the present anti-stick agents include homopolymers of alkyl methicone, phenyl methicone, dimethicone, alkyl trimethicone, phenyl trimethicone, polyol, polyether (e.g., polyoxymethylene, polyoxyethylene and polyoxypropylene), polyimine, polyamide, polyacrylate, polyester, and copolymers containing one or multiple of these polymeric units. Non-limiting examples of suitable polymers include polydimethyl siloxane, polyethylimine, polyacrylic acid, poly(ethylene-co-acrylic acid), polymethacrylic acid, poly(ethylene-co-methacrylic acid), poly(vinyl acetate), polyvinylpyrrolidone, poly(ethylene-co-vinyl acetate), poly(butanediol), poly(neopentyl glycol), poly(ethylene adipate), poly(butylene adipate), poly(ethylene glutamate), poly(butylene glutamate), poly(ethylene sebacate), poly(butylene sebacate), poly(ethylene succinate), poly(butylene succinate), poly(ethylene terephthalate), poly(butylene terephthalate), polycaprolactone, and polyglycerol.

Non-limiting examples of suitable PPG copolymer materials include PPG-12 dimethicone such as Sisoft 910 (Momentive); bis-PPG-15 dimethicon/IPDI copolymer such as Polyderm-PPI-SI-WI (Alzo); PPG/polycaprolactone block copolymer; PPG/polybutanediol/PEG triblock copolymer; polyethylimine/PPG copolymer and polyacrylic acid-g-PPG graft copolymer.

Another suitable PPG material includes PPG surfactant materials. The following formula represents suitable PPG surfactant materials wherein the PPG segments constitute a part of the head functional group:

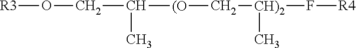

wherein R3 is hydrogen, alkyl, alkyl carbonyl, alkylenelamine, alkylenelamide, alkylene phosphate, alkylene carboxylic acid, alkylene sulfonate salt and alkylene quat with the maximum number of carbon element less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100, alternatively from 2 to 30, and alternatively from 3 to 25; and F is a functional group selected from the group of ether groups (including oxy, glyceryl, glucose, sorbitan, sucrose, monoethanolamine or diethanolamine), ester groups (including ester, glyceryl ester, glucose ester, sorbitan ester or sucrose ester), amine groups, amide groups, and phosphate ester groups.

The following formula represents suitable PPG surfactant materials wherein the PPG segments constitute an internal block group:

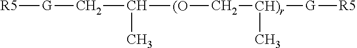

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120, alternatively from 4 to 50, and alternatively from 6 to 30; and G is ether, ester, amine, or amide linkage.

Non-limiting examples of suitable PPG surfactant materials include PPG-30 cetyl ether such as Hetoxol C30P (Global Seven); PPG-20 methyl glucose ether distearate such as Glucam P-20 Distearate Emollient (Noveon), PPG-20 methyl glucose ether acetate, PPG-20 sorbitan tristearate, PPG-20 methyl glucose ether distearate, PPG-20 distearate, PPG-15 stearyl ether such as Alamol-E (Croda-Uniqema) and Procetyl 15 (Croda), PPG-15 stearyl ether benzoate, PPG-15 isohexadecyl ether, PPG-15 stearate, PPG-15 dicocoate, PPG-12 dilaurate, PPG-11 stearyl ether such as Varonic APS (Evonik); PPG-10 cetyl ether such as Procetyl 10 (Croda); PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether such as Promyristyl PM-3 (Croda), PPG-3 myristyl ether propionate such as Crodamol PMP (Croda), PPG-3 benzyl ether myristate such as Crodamol STS (Croda), PPG-3 hydrogenated castor oil such as Hetester HCP (Alzo), PPG-3-hydroxyethyl soyamide, PPG-2 Cocamide, PPG-2 lanolin alcohol ether and PPG-1 coconut fatty acid isopropanolamide such as Amizett IPC (Kawaken Fine Chemicals). Suitable PPG material is PPG-15 stearyl ether, such as the product sold as CETIOL E, by BASF Corporation (Florham Park, New Jersey, USA) and/or BASF SE (Ludwigshafen, Germany).

The anti-stick agents contemplated herein may include the carrier at a total carrier concentration ranging from about 60% to 99.9%, alternatively about 70% to 99.5%, alternatively about 80% to 99% by weight of the anti-stick agent.

Carriers suitable herein may include oils or fats such as natural oils or fats, or natural oil or fat derivatives, in particular of plant or animal origin. Non-limiting examples include avocado oil, apricot oil, apricot kernel oil, babassu oil, borage oil, borage seed oil, calendula oil, camellia oil, canola oil, carrot oil, cashew nut oil, castor oil, chamomile oil, cherry pit oil, chia oil, coconut oil, cod liver oil, corn oil, corn germ oil, cottonseed oil, eucalyptus oil, evening primrose oil, grape seed oil, hazelnut oil, jojoba oil, juniper oil, kernel oil, linseed oil, macadamia oil, meadowfoam seed oil, menhaden oil, mink oil, moringa oil, mortierella oil, olive oil, palm oil, palm kernel oil, peanut oil, peach kernel oil, rapeseed oil, rose hip oil, safflower oil, sandlewood oil, sesame oil, soybean oil, sunflower oil, sunflower seed oil, sweet almond oil, tall oil, tea tree oil, turnip seed oil, walnut oil, wheat germ oil, zadoary oil, or the hardened derivatives thereof. Hardened oils or fats from vegetal origin can include, e.g., hardened castor oil, peanut oil, soya oil, turnip seed oil, cottonseed oil, sunflower oil, palm oil, kernel oil, linseed oil, corn oil, olive oil, sesame oil, cocoa butter, shea butter and coconut oil.

Other non-limiting examples of fats and oils may include: butter, C12-C18 acid triglyceride, camellia oil, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, human placental lipids, hybrid safflower oil, hybrid sunflower seed oil, hydrogenated castor oil, hydrogenated castor oil laurate, hydrogenated coconut oil, hydrogenated cottonseed oil, hydrogenated C12-C18 triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lanolin and lanolin derivatives, lanolin alcohol, lard, lauric/palmitic/oleic triglyceride, lesquerella oil, maleated soybean oil, meadowfoam oil, neatsfoot oil, oleic/linoleic triglyceride, oleic/palmitic/lauric/myristic/linoleic triglyceride, oleostearine, olive husk oil, omental lipids, orange roughy oil, pengawar djambi oil, pentadesma butter, phospholipids, pistachio nut oil, placental lipids, rapeseed oil, rice bran oil, shark liver oil, shea butter, sphingolipids, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, wheat bran lipids, and the like, as well as mixtures thereof. Suitable carriers include caprylic/capric triglyceride. This material is currently available as, e.g., MYRITOL 318, a product of BASF Corporation (Florham Park, New Jersey, USA) and/or BASF SE (Ludwigshafen, Germany).

Other suitable carriers may include mono- or di-glycerides, such as those derived from saturated or unsaturated, linear or branch chained, substituted or unsubstituted fatty acids or fatty acid mixtures. Examples of mono- or diglycerides include mono- or di-C12-24fatty acid glycerides, specifically mono- or di-C16-20fatty acid glycerides, for example glyceryl monostearate, glyceryl distearate. Carriers may also include esters of linear C6-C22-fatty acids with branched alcohols. Carriers contemplated herein may also include sterols, phytosterols, and sterol derivatives. Sterols and sterol derivatives that can be used in the anti-stick agents of the invention include, but are not limited to: B-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

### Fluid Management Layer

The fluid management layer 30 adds caliper to the absorbent article and is typically compressible, and resilient, which can impart a feeling of softness and/or a “cushiony” feel to the article. The absorbent articles contemplated herein exhibit good resiliency properties both in dry and wet conditions. The fluid management layer described herein is a nonwoven and comprises cellulosic fibers, divider fibers, and bonding fibers. At least two of the cellulosic fibers, divider fibers, and bonding fibers have a decitex of no greater than 2 or less than about 2. For example, in some embodiments, the cellulosic fibers and the bonding fibers have a decitex of no greater than about 2. The fluid management layer 30 has a caliper factor (mm per 10 gsm) of from about 0.12 to about 0.40, as determined by the Caliper Factor test method disclosed herein. To maintain this relatively high caliper, the fluid management layer must include finer fibers (decitex less than about 2) to maintain the desired pore size (from about 40 μm to about 150 μm or from about 50 μm to about 120 μm or from about 60 μm to about 100 μm), so that fluid is quickly transferred to the absorbent core and is trapped within the core to prevent rewet. Because of these finer fibers (decitex less than about 2) and the need to maintain caliper or thickness of the layer, needlepunching is an ideal process because it does not as aggressively adversely affect the caliper. The needles are able to move and integrate the finer fibers without compressing the layer, which allows the caliper to be maintained.

The fluid management layer 30 may include a nonwoven having a basis weight of from about 40 to about 85 gsm. The fluid management layer 30 may have a basis weight of up to 75 grams per square meter (gsm); or a basis weight of up to 70 gsm; or a basis weight in the range of about 40 gsm to about 75 gsm; or in the range of about 50 gsm to about 70 gsm; or in the range of about 55 gsm to about 65 gsm, including any values within these ranges and any ranges created thereby. The fluid management layer may be any suitable shape including but not limited to oval, a stadium, rectangle, an asymmetric shape, peanut, trapezoid, rounded trapezoid, ovoid, nested, and/or hourglass. In some examples, the fluid management layer may have a contoured shape, e.g., one that is narrower in the longitudinally intermediate region than in the end regions. In other examples, the fluid management layer may have a tapered shape that is a wider in one end region of the pad, and tapers to a narrower width in the other end region of the pad. The fluid management layer may have a long oval shape. The fluid management layer may have a nested shape where one end is concave, and the other end is convex.

In addition to the softness and resiliency benefits of compositions and structures for fluid management layers contemplated herein, stain size control and faster fluid acquisition may be obtained. Stain size is important in the way the absorbent article is perceived by the user. For feminine hygiene pads, when a stain visible on the pad after a duration of use/wear is relatively large along x-y directions, users may perceive that the pad is near failure based on the appearance of the stain and its proximity to the outer periphery of the pad. In contrast, a smaller, lighter stain can have a reassuring effect on the user/wearer, by creating a perception that the pad is not near failure because the edges of the stain lie substantially longitudinally and/or laterally short of the outer periphery of the pad. The ability of the fluid management layer to pull fluid toward the garment-facing surface and away from the wearer-facing surface results in relatively reduced stain size. The structure of the fluid management layer discussed herein allows for fluid to be more readily pulled away from the wearer-facing surface.

Additionally, fluid acquisition speed of the absorbent article may be deemed important to the user/wearer, as rapid acquisition can help make the user/wearer feel dry and clean. When the absorbent article requires a relatively long time to drain discharged fluid from the topsheet, it can cause the user to feel wetness, and feel unclean. To enable rapid acquisition/intake of discharged fluid, but also sufficient capillarity to dewater the topsheet, the fluid management layer has a particular pore volume range. The fluid management layer has an average pore size of from about 40 to about 330 μm, or, alternatively, the average pore size may be from about 40 μm to about 150 μm or from about 50 μm to about 120 μm or from about 60 μm to about 100 μm, as determined by the Micro-CT Pore Size Measurement Method. The mean pore size of the fluid management layer is less than the mean pore size of the topsheet to control the acquisition and transfer of fluid to the absorbent core.

The fluid management layer can draw fluid through and from the topsheet via capillary action or wicking forces, of sufficient magnitude to overcome any resistance to passage of the fluid through the topsheet, or attraction the topsheet may have for the fluid, that may be present as a result of the composition and/or configuration of the topsheet. The fluid management layer also can accept and contain a gush of fluid by providing pore volume as a temporary reservoir, together with distribution functions, to efficiently utilize the absorbent structure, give it time to imbibe and absorb the fluid.

The inventors have found that to deliver the desired levels of softness and resiliency combined with the need for rapid acquisition and high capillarity needs, that the fluid management layer breaks technology limitations of the past. The fluid management layer of the present invention delivers high caliper, that is resilient and yet flexible combined with high capillarity and permeability.

Overall, the fluid management layer of the present disclosure may comprise from about 15 percent to about 40 percent by weight, from about 20 percent to about 35 percent by weight, from about 20 percent to about 30 percent by weight of the fluid management layer of cellulose fibers, specifically including any values within these ranges and any ranges created thereby. In one specific example, fluid management layers may comprise about 20 percent by weight of cellulosic fibers. Suitable cellulosic fibers include cotton, rayon, viscose, lyocell, natural cellulose, regenerated cellulose, and combinations thereof. Particularly suitable cellulosic fibers include viscose. The cellulosic fibers have a decitex of less than about 2, or, alternatively, from about 0.5 to about 1.7.

The cellulosic fibers of the fluid management layer may have any suitable cross-section profile shape (where the cross-section lies along a plane that is perpendicular with the greater length dimension of the fiber when it is straight). Some examples of suitable shapes may include trilobal, “H,” “Y,” “X,” “T,” round, or flat ribbon. Further, the absorbing fibers can have cross sections that are solid, hollow, or combinations of hollow and solid. Other examples of suitable multi-lobed, cellulosic fibers for utilization in the fluid management layers described herein are disclosed in U.S. Pat. Nos. 6,333,108; 5,634,914; and 5,458,835. A trilobal fiber shape can improve wicking and improve opacity and stain concealment properties. Suitable trilobal rayon fibers are available from Kelheim Fibres GmbH (Kelheim, Germany) and sold under the trade name GALAXY. While each stratum may include a different shape of cellulosic fiber, much like mentioned above, not all carding equipment may be suited to handle such variation between/among strata. In one specific example, the fluid management layer may include cellulosic fibers having a round (circular) shape.

The staple length of the cellulosic fibers may be selected to be about 20 mm to about 100 mm, or about 30 mm to about 50 mm, or from about 35 mm to about 45 mm, specifically reciting all values within these ranges and any ranges created thereby. In general, the fiber length of wood pulp is from about 4 to about 6 mm and cannot be used in conventional carding machines because the pulp fibers are too short. Accordingly, if wood pulp is desired as a fiber in the fluid management layer, additional processes to blend and add pulp to the carded webs may be beneficial. In some examples, pulp may be airlaid between carded webs with the combination being subsequently integrated. As another example, tissue made from pulp may be utilized in combination with the carded webs and the combination may be subsequently integrated.

Similarly, overall, the fluid management layers of the present disclosure may comprise from about 20 percent to about 40 percent by weight of the fluid management layer of bonding fibers, specifically reciting all values within these ranges and any ranges created thereby. Suitable bonding fibers include bicomponent polyethylene terephthalate/polyethylene, combinations of polyethylene, polypropylene, polyethylene terephthalate, Co-polyethylene terephthalate and combinations thereof. The bonding fiber can be polyethylene terephthalate/polyethylene wherein the core is polyethylene terephthalate, and the sheath is polyethylene. The bonding fibers may comprise bicomponent fibers. Particularly suitable bonding fibers may comprise polymeric fibers. The bonding fibers may comprise shape memory fibers, which are discussed in Shape-Memory Polymers, Angew. Chem. Int. Ed. 2002, 41, 2034-2057, and Recent advances in shape memory polymers and composites: a review, J Mater Sci (2008) 43:254-269. Shape memory fibers are those fibers that have the ability to recover to their original shape after undergoing compression, for example. The shape memory fibers may include polyethylene terephthalate, polytrimethylene terephthalate, polybutylene, polyethylene terephthalate copolymer, poly(tetramethylene ether) glycol, and combinations. Shape memory fibers are discussed in US 2022/0133552 filed Oct. 26, 2021 and PCT/CN2024/105604 filed on Jul. 16, 2025, which are each incorporated by reference. The bonding fibers have a decitex of less than about 2, or, alternatively, from about 1 to about 2. The bonding fibers enhance the ability of the fluid management layer to recover its shape and/or caliper following application of compressive loads that are imposed during use. Stated another way, the bonding fibers provide resiliency to the fluid management layer.

The second sublayer may also comprise from about 40 weight percent to about 60 weight percent of the fluid management layer of divider fibers, or from about 50 weight percent to about 55 weight percent of the fluid management layer of divider fibers. Suitable divider fibers include polypropylene, polyethylene terephthalate, bicomponent polyethylene, bicomponent polypropylene, bicomponent polyethylene terephthalate and combinations thereof. Suitable divider fibers have a decitex of less than about 2, alternatively from about 0.5 to about 2. Particularly suitable divider fibers may comprise non-cylindrical polymeric fibers including but not limited to polypropylene. The divider fibers function to divide spaces in between the bonding and cellulosic fibers thereby creating smaller pore sizes that drive capillarity. The small size and optional non-cylindrical shape further enhance the capillarity.

The cellulose, bonding and/or divider fibers can have a length of from about 10 to about 120 mm, alternatively from about 24 to about 95 mm, and alternatively from about 36 to about 75 mm. The cellulose, bonding, and divider fibers each have a fiber length and the fiber lengths of each of these types of fibers may be the same length, a different length, or a combination thereof. The weight fractions of cellulosic fibers, bonding fibers, and/or divider fibers may be determined via the Material Compositional Analysis method disclosed below.

In order to deliver the needed nonwoven caliper and the necessary pore structure for fluid performance it is necessary to utilize low decitex fibers as previously disclosed. It is additionally important to utilize a process that imparts material strength in both the MD and CD directions. This is particularly difficult as improper integration methods of the fibers will collapse the overall material caliper, especially since the fiber decitex are so low. The fluid management layer comprises integrated stitches at a stitch density of between about 50 to about 250 punches per square centimeter or from about 90 to about 200 punches per square centimeter. The stitch direction is selected from the top, bottom, and combinations thereof. More specifically, the plurality of needles may penetrate into the layer from the top surface and move toward the bottom surface (a top stitch direction), or the plurality of needles may penetrate into the layer from the bottom surface and move toward the top surface (a bottom stitch direction), or a first portion of the plurality of needles may engage the layer in a top stitch direction and a second portion of the plurality of needles may engage the layer in a bottom stitch direction. Additionally, the fluid management layer has an MD: CD fiber orientation from about 1:1 to about 1:1.75.

The fluid management layer can have a MD peak load of from about 4 to about 85 Newtons and may also have a CD peak load of from about 4 to about 130 Newtons. The fluid management layer 30 can have a caliper factor (mm per 10 gsm) of between 0.12 to about 0.40, including all values within these ranges and any ranges created thereby. More specifically, the fluid management layer 30 may have a caliper factor measured prior to winding the web or substrate used to form the fluid management layer and after the web of material used to form the fluid management layer ifs formed of from about 0.26 to about 0.40. Additionally, the fluid management layer may have a caliper factor measured after unwinding the web or substrate and/or after cutting the web or substrate to form the individual fluid management layers to be included in the absorbent article of from about 0.12 to about 0.25. The difference in the caliper factor of the fluid management layer is due to the processing the fluid management layer undergoes from web/substrate formation to inclusion in the final absorbent article. For example, by winding the fluid management layer web/substrate, tension is placed on the web and the caliper may be adversely affected by the strain and compression placed on the web/substrate. However, it is to be appreciated that the caliper factor of the fluid management layer as placed in the absorbent article may be the same as the caliper factor of the fluid management layer substrate/web after formation if the additional processes do not adversely affect the fluid management layer caliper. Thus, it is recognized that the caliper factor (mm per 10 gsm) is process dependent and may be from about 0.12 to about 0.40 measured at any point from formation to inclusion into the final product. The caliper and caliper factor of the fluid management layer of the present disclosure may be determined by the Caliper and Caliper Factor test methods disclosed herein.

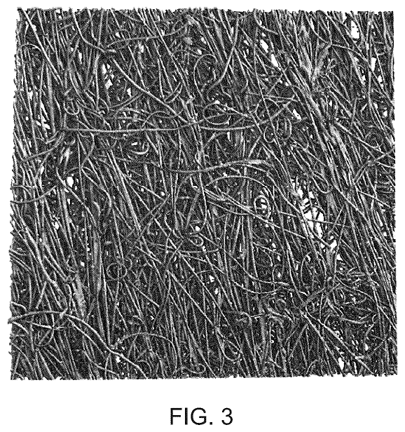

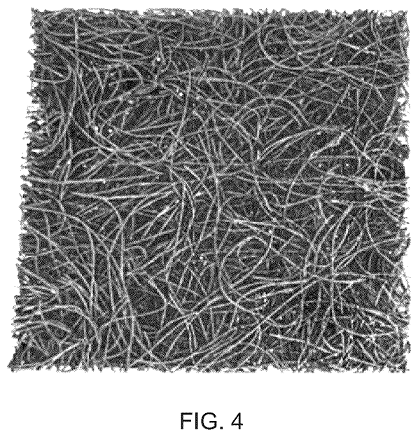

The inventors have found that the material can be both crosslapped and integrated via the use of specially crafted needles. This delivers the desired MD and CD material tensiles (reported as peak load) and MD/CD tensile ratios and maintains the overall material caliper as the energy to entangle is concentrated to specific fibers vs the entire web. Shown in FIG. 3 is a non-crosslapped fluid management layer. Shown in FIG. 4 is a crosslapped fluid management layer. In FIG. 4 the fibers are oriented in both the MD and CD directions.

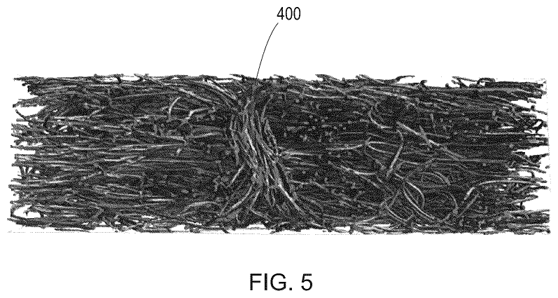

Needling reorients fibers from the x-y plane to the z-direction to create concentrated bundles of fibers oriented in the z-direction (as shown in FIG. 5). The vertical bundles create pathways for fluid to flow efficiently through the material in the z-direction to reach the core faster, particularly in gush situations. The vertical fiber bundles 400 also increase resilience and compression resistance in the z-direction.

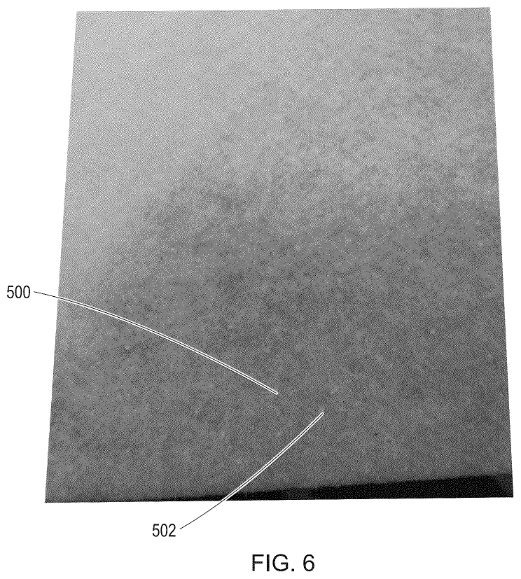

To further increase the ability of the fluid management layer to dry the topsheet, the fluid management layer can have small highly concentrated fiber areas spread throughout the fluid management layer. These small highly concentrated fiber areas, or capillary boosting points 500, 502 can be seen in FIG. 6. The capillarity boosting points have higher capillarity but as they are spread out over a wide area, they have minimal, or no, impact on overall permeability and hence minimal, or no, impact on the acquisition speed of the topsheet. These capillary boosting points can be spaced apart in various ways. For instance, there can be from about 1 capillary boosting point to about 10 capillary boosting points per square inch; alternatively, from about 3 to about 7 capillary boosting points per square inch. The capillary boosting points can be easily seen optically, especially with the use of a light table where the number of points is counted within a square inch of the material. The capillarity boosting points can vary in size from about 0.5 mm² to about 5 mm². The creation of the Capillary boosting points is controlled via careful calibration of the speeds within the carding unit and the speed of the needle punches combined with the small decitex fiber choices described herein.

The nonwoven of the present invention is initially fiber blended, accumulated and laydown and fed through one or more carding steps. The non-woven material is then cross-lapped prior to the web forming process step. Crosslapping is well known to a person skilled in the art. For example, the carded web material is moved forward and backwards when laid on a belt or carrier while its lower front portion is pulled perpendicular to this forward and backward movement whereby the web material overlaps in a z-like fashion. This imparts a sufficient MD/CD tensile ratio.

The non-woven is then needlepunched, with the aid of one or more needle looms. Here a web of loose fibers, e.g., a web of carded fibers, is converted into a coherent non-woven fabric. The needlepunched fibers are mechanically oriented through the web. The needles can be arranged on a needle tool, e.g., a needle board or loom, in a non-lined arrangement. In the needle punching step at least one needle tool can be used in the range of from about 50 to 250, alternatively from about 70 to 200, and alternatively from about 90 to 180 needles per square inch.

The non-woven is then bonded via heat treatment after needling of the fluid management layer material. This bonding of the bonding fibers creates a support matrix which enhances resiliency and stiffness of the fluid management layer.

Absorbent articles that exhibit a soft cushiony feel, good resiliency and fluid handling characteristics are contemplated herein. Toward imparting these characteristics, the caliper of the fluid management layer may be deemed important. Typical calipers of webs from conventional spunlace lines achieve a caliper factor (caliper per 10 gsm of basis weight) of 0.03 mm/gsm to 0.12 mm/gsm. In contrast, the fluid management layers contemplated herein can exhibit a caliper factor (mm per 10 gsm) of greater than 0.12 or greater than about 0.15 or at least 0.26, or from about 0.15 to about 0.40, including all values within these ranges and any ranges created thereby. The fluid management layers contemplated herein can have a caliper factor of between 0.26 to about 0.40, including all values within these ranges and any ranges created thereby. It is important to note that the caliper factors mentioned heretofore are with regard to caliper obtained using the Caliper Factor test method as disclosed herein.

In the heat bonding process, the heating temperature selection may be impacted, in part, by the constituent composition(s) of the bonding fibers, the design and operating parameters of the heating equipment, and the web processing speed (i.e., duration of exposure to the heated environment). To impart uniform stiffness across the fluid management layer, the heating equipment and operating parameters should be set up to provide uniform heating to the fluid management layer web. Even small variations in temperature can substantially impact the formation of fiber-to-fiber bonds between the bonding fibers and resulting tensile strength of the fluid management layer. An example of a suitable heat stiffening process that may be utilized is air-through heating, in which air heated to the selected heating temperature is blown and/or drawn (via vacuum) through the web along a direction that is approximately orthogonal to the larger planes defined by the web. Suitable MD/CD Peak Load ratios range from about 0.5 to 1.75.

In order to enhance the stabilizing effect of the integration, crimped fibers may be utilized. As discussed in additional detail below, the fluid management layers of the present disclosure may comprise cellulose fibers, bonding fibers, and additionally may comprise divider fibers. One or more of these fibers may be crimped prior to integration. For example, where synthetic fibers are utilized, these fibers may be mechanically crimped via intermeshing teeth. And for the cellulosic fibers, these fibers may be mechanically crimped and/or may have a chemically induced crimp due to the variable skin thickness formed during creation of the cellulosic fibers.

Fluid management layers contemplated herein may be incorporated into a variety of absorbent articles. A non-limiting example of a schematic representation of an absorbent article in the form of a feminine hygiene pad as contemplated herein is shown in FIG. 1. As reflected, the pad 10 as contemplated herein may include a topsheet 20, a backsheet 50, and an absorbent structure 40 disposed between the topsheet 20 and the backsheet 50. A fluid management layer 30 may be disposed between the topsheet 20 and the absorbent structure 40. The pad has a wearer-facing surface 62 and an opposing outward-facing surface 64. The wearer-facing surface 62 is formed primarily by the topsheet 20 while the outward-facing surface 64 is formed primarily by the backsheet 50. Additional components (not shown) may be included proximate the wearer-facing surface 62 and/or the outward-facing surface 64. For example, if the absorbent article is an incontinence pad, a pair of barrier cuffs which extend generally parallel to a longitudinal axis of the pad 10 and may also form portions of the wearer-facing surface 62. Similarly, one or more deposits fastening adhesive (to be used by the user/wearer to affix the pad within her underwear, at an appropriate location, for use) may be present on the backsheet 50 and form a portion of the outward-facing surface 64 of the absorbent article.

### Absorbent Structure

The absorbent structure 40 of the present disclosure may have any suitable shape including but not limited to oval, a stadium, rectangle, an asymmetric shape, peanut, trapezoid, rounded trapezoid, ovoid, nested and hourglass. In some examples, absorbent structure 40 may have a contoured shape, e.g., one that is narrower in the longitudinally intermediate region than in the end regions. In other examples, the absorbent structure may have a tapered shape that is a wider in one end region of the pad, and tapers to a narrower width in the other end region of the pad. The absorbent structure may have a nested shape where one end is concave, and the other end is convex. The absorbent structure 40 may have varying stiffnesses in the MD and CD.

The configuration and construction of the absorbent structure 40 may vary (e.g., the absorbent structure 40 may have varying caliper zones, a hydrophilic gradient, a superabsorbent gradient, or lower average density and lower average basis weight acquisition zones). Further, the size and absorbent capacity of the absorbent structure 40 may also be varied to accommodate a variety of wearers. However, the total absorbent capacity of the absorbent structure 40 should be compatible with the design loading and the intended use of the disposable absorbent article or incontinence pad 10.

In some examples, the absorbent structure 40 may include a plurality of layers each having particular features and/or functions. Are some examples, the absorbent structure 40 may include a wrap (not shown) included to envelope enveloping the absorbent constituents of the absorbent structure. The wrap may be formed by one or more nonwoven materials, tissues, films or other materials, or laminates thereof. In one form, the wrap may be formed of only a single material, substrate, laminate, or other material that is wrapped at least partially around itself.

The absorbent structure 40 may include one or more adhesives, for example, to help immobilize the SAP or other absorbent materials within the first and second laminates.

Suitable absorbent structures comprising relatively high amounts of superabsorbent polymer (“SAP”—also known as “absorbent gelling material,” or “AGM”) with various core designs are disclosed in U.S. Pat. No. 5,599,335; EP 1 447 066; WO 95/11652; US 2008/0312622 A1; and WO 2012/052172

Additions to the absorbent structure are contemplated. Potential additions to the absorbent structure are described in U.S. Pat. Nos. 4,610,678; 4,673,402; 4,888,231; and 4,834,735. The absorbent structure may further include layers that mimic the dual core system containing an acquisition/distribution core of chemically stiffened fibers positioned over an absorbent storage core as described in U.S. Pat. No. 5,234,423; and in U.S. Pat. No. 5,147,345. These may be deemed useful to the extent they do not negate or conflict with the effects of the below described laminates of the absorbent structure of the present invention.

Some further examples of a suitable absorbent structures 40 that can be used in the absorbent article of the present disclosure are described in US 2018/0098893 and US 2018/0098891.

As noted above, absorbent articles including a fluid management layer contemplated herein may include a storage layer. Referring back to FIGS. 1 and 1A, a storage layer would generally be positioned at a location corresponding to that in which the absorbent structure 40 is depicted. The storage layer may be constructed as described regarding absorbent structures. The storage layer may contain conventional absorbent materials. In addition to conventional absorbent materials such as creped cellulose wadding, fluffed cellulose fibers, rayon or viscose fibers and comminuted wood pulp fibers (also known as airfelt or fluff pulp), and textile fibers, the storage layer may also include particles or fibers of superabsorbent material that imbibes fluids and forms hydrogels. (Such materials are also known as absorbent gelling materials (AGM).) AGM is typically capable of absorbing a relatively large weight quantity of body fluid per dry weight AGM, retaining it under moderate pressure. Synthetic fibers spun from polymers such as cellulose acetate, polyvinyl fluoride, polyvinylidene chloride, acrylics (such as ORLON), polyvinyl acetate, non-soluble polyvinyl alcohol, polyethylene, polypropylene, polyamides (such as nylon), polyesters, bi-component fibers, tricomponent fibers, mixtures thereof and the like can also be included in the secondary storage layer. The storage layer may also include filler materials, such as PERLITE, diatomaceous earth, VERMICULITE, or other suitable materials, that can serve to reduce changes of rewetting.

The storage layer or fluid storage layer may include absorbent gelling material (AGM) in a uniform distribution throughout or may include it in a non-uniform distribution. The AGM may be distributed and/or concentrated via deposit thereof into channels or pockets, or may be deposited in patterns including stripes, crisscross patterns, swirls, dots, or any other pattern, either two or three dimensional, that can be imagined. The AGM may be sandwiched between a pair of fibrous cover layers. AGM may be encapsulated, at least in part, by a single fibrous cover layer.

Portions of the storage layer may be formed substantially only of superabsorbent material/AGM, or may be formed of AGM distributed and dispersed in a suitable carrier structure such as a batt or accumulation of cellulose fibers in the form of fluff or stiffened fibers. One non-limiting example of a storage layer may include a first layer formed substantially only of AGM particles or fibers, that are placed or deposited onto a second layer that is formed of a distribution of AGM particles or fibers, within cellulose fibers.

Examples of absorbent structures formed of layers of superabsorbent material/AGM and/or layers of superabsorbent material/AGM dispersed within a batt or other accumulation of cellulose fibers, that may be utilized in the absorbent articles (e.g., sanitary napkins, incontinence products) contemplated herein are disclosed in US 2010/0228209A1. Absorbent structures comprising relatively high amounts of SAP/AGM with various core designs are disclosed in U.S. Pat. No. 5,599,335; EP 1 447 066; WO 95/11652; US. 2008/0312622 A1; WO 2012/052172; U.S. Pat. Nos. 8,466,336; and 9,693,910 to Carlucci. These may be used to configure the absorbent structure or storage layer.

### Backsheet

The backsheet 50 may be disposed beneath the absorbent structure 40 and be the outwardmost layer of the article, forming the outward-facing surface of the article. The backsheet 50 may be joined to the absorbent structure 40 and/or to the topsheet (about the outer periphery) by any suitable attachment methods known in the art. For example, the backsheet 50 may be secured to the absorbent structure 40 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet may be impervious, or substantially impervious, to liquids (e.g., urine, menstrual fluid) under ordinary conditions of use, and may be manufactured from a thin plastic film, although other flexible liquid impervious materials may also be used. The backsheet may prevent, or at least inhibit, exudates absorbed and contained in the absorbent structure from wetting underwear, outer clothing, bedding, etc. which may come into contact with or proximity to the article. However, in some examples the backsheet may be configured so as permit vapor to escape from the absorbent structure (i.e., is “breathable”) while in examples the backsheet may be configured so as to be vapor-impermeable (i.e., non-breathable). Backsheet may include a polymeric film such as a film of polyethylene or polypropylene. A suitable material for the backsheet is a thermoplastic film having a thickness of approximately 0.012 mm (0.5 mil) to 0.051 mm (2.0 mils), for example. Suitable materials for the backsheet film may have a basis weight of from about 8 to about 25 gsm. Any suitable liquid impermeable backsheet known in the art may be utilized with the present invention.

The backsheet serves as a barrier to prevent migration of fluids absorbed and retained in the absorbent structure, to the outward-facing surface of the pad. Suitable materials are soft, smooth, compliant, liquid and vapor pervious material that provides for softness and conformability for comfort and is low noise producing so that movement does not cause unwanted sound.

Non-limiting examples of materials suitable for forming backsheets are described in U.S. Pat. Nos. 5,885,265; 6,462,251; 6,623,464; and 6,664,439. Examples of suitable dual- or multi-layer breathable backsheets include those described in U.S. Pat. Nos. 3,881,489; 4,341,216; 4,713,068; 4,818,600; EP 203 821; EP 710 471; EP 710 472; and EP 793 952. Additional examples of suitable single layer breathable backsheets for include those described in GB A 2184 389; GB A 2184 390; GB A 2184 391; U.S. Pat. Nos. 4,591,523; 3,989,867; 3,156,242; and WO 97/24097.

The backsheet may be a nonwoven web having a basis weight of about 20 gsm to 50 gsm. In one example, the backsheet may be a hydrophobic 23 gsm spunbond nonwoven web of 4 denier polypropylene fibers, available from Fiberweb Neuberger, under the trade designation F102301001. The backsheet may be coated with a non-soluble, liquid swellable material as described in U.S. Pat. No. 6,436,508.

The backsheet has an outward-facing side and an opposing wearer-facing side. The outward-facing side of the backsheet may include a non-adhesive area and an adhesive area. The adhesive area may be provided by any conventional means, for the purpose of enabling the user/wearer to affix the pad to the wearer-facing surface of her underwear at a location suitable for use. Pressure-sensitive adhesives have been found to work well for this purpose.

Traditional absorbent articles extend the width of the core in the crotch area as far as possible to acquire fluid that may escape to the edges. This can create an uncomfortable experience for the wearer. Other traditional absorbent articles narrow the core to improve comfort and attempt to extend the fluid management layers into wider areas in the crotch, but this does not create the necessary distance or changes in topography needed for best performance.

An additional feature of the absorbent article described herein is a stepped side barrier sufficient to capture fluid that may escape to the edges of the absorbent article. The disposable absorbent article comprises a topsheet, a backsheet, an absorbent core disposed between the topsheet and the backsheet, and a fluid management layer disposed between the topsheet and the absorbent core. The fluid management layer described herein is combined with the topsheet in the process to create a differential tension composite web. This enables the topsheet to conform sufficiently around the side edges of the fluid management layer and then to be bonded to the backsheet while preserving the topographical features of the fluid management layer on the edges of the fluid management layer in the crotch area. The fluid management layer is wider than the absorbent core edge in the crotch area thereby creating a flexible and comfortable product while preserving a minimum of about 1.5 mm of step change in the crotch area. Suitable ranges for step change are from about 1.8 mm to about 3.5 mm.

### Examples and Data

As previously discussed, the fluid management layer as discussed herein exhibits exemplary fluid handling properties in comparison to other, com fluid management layers produced by alternate methods and/or including fibers of a generally higher decitex. The inventive samples are produced using a needlepunch process resulting in the fluid management layer including a plurality of vertical fiber bundles 400, as previously discussed, that increase resilience and compression resistance in the z-direction. The following examples include inventive samples and their respective data.

Inventive Sample 1 includes a fluid management layer having a basis weight of 71.68 gsm and having 25 percent by weight viscose cellulose fibers having a 1.3 dtex; 75 percent by weight bi-component fibers having a 1.7 dtex. The bi-component fibers have a polyethylene terephthalate component and polyethylene component in a core-sheath configuration, where the polyethylene is the sheath. The fluid management layer includes a plurality of vertical fiber bundles having a stitch density of 150 punches per square centimeter.

Inventive Sample 2 includes a fluid management layer having a basis weight of 65.61 gsm and having 25 percent by weight cellulose fibers having a 0.9 dtex; 45 percent by weight tri-lobal polypropylene fibers having a 1.0 dtex; and 30 percent by weight bi-component fibers having a 1.7 dtex. The cellulosic fibers are viscose fibers. The bi-component fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter.

Inventive Sample 3 includes a fluid management layer having a basis weight of 64.98 gsm and having 25 percent by weight cellulose fibers having a 0.9 dtex; 45 percent by weight tri-lobal polypropylene fibers having a 1.0 dtex; and 30 percent by weight bi-component fibers at 1.7 dtex. The cellulose fibers are viscose fibers. The bi-component fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter.

Inventive Sample 4 includes a fluid management layer having a basis weight of 57.54 gsm and having 25 percent by weight cellulose fibers having a 0.9 dtex; 45 percent by weight tri-lobal polypropylene fibers having a 1.0 dtex; 30 percent by weight bi-component fibers having a 1.7 dtex. The cellulose fibers are viscose fibers. The bicomponent fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter.

Inventive Sample 5 includes a fluid management layer having a basis weight of 62.00 gsm and having 25 percent by weight cellulose fibers having a 0.9 dtex; 45 percent by weight tri-lobal polypropylene fibers having a 1.0 dtex; 30 percent by weight bi-component fibers having a 1.7 dtex. The cellulose fibers are viscose fibers. The bicomponent fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter.

Comparative Sample 1 includes a fluid management layer produced by a spunlace process and, therefore, does not include any vertical fiber bundles. The comparative sample 1 fluid management layer having a basis weight of 68.99 gsm and having 40 percent by weight cellulose fibers having a 1.7 dtex; 40 percent by weight bicomponent fibers having a 1.7 dtex; and 20 percent by weight of polyethylene terephthalate fibers having a 4.4 dtex. The cellulose fibers are viscose fibers. The bicomponent fibers included a first component polyethylene terephthalate and a second component polyethylene in a core-sheath configuration, where the polyethylene is the sheath.

Comparative Sample 2 includes a fluid management layer produced by a spunlace process and, therefore, does not include any vertical fiber bundles. The comparative sample 2 fluid management layer having a basis weight of 65.4 gsm and having 20 percent by weight cellulose fibers having a 1.3 dtex; 30 percent by weight bicomponent fibers having a 10 dtex; and 50 percent by weight of polyethylene terephthalate fibers having a 2.2 dtex. The cellulose fibers are viscose fibers. The bicomponent fibers included a first component polyethylene terephthalate and a second component polyethylene in a core-sheath configuration, where the polyethylene is the sheath.

Comparative Sample 3 includes a fluid management layer produced by a spunlace process and, therefore, does not including any vertical fiber bundles. The comparative sample 3 fluid management layer having a basis weight of 58.5 gsm and having 40 percent by weight cellulose fibers having a 1.7 dtex; 25 percent by weight bicomponent fibers having a 2.2 dtex; and 35 percent by weight of polyethylene terephthalate fibers having a 1.7 dtex. The cellulose fibers are viscose fibers. The bicomponent fibers included a first component polyethylene terephthalate and a second component polyethylene in a core-sheath configuration, where the polyethylene is the sheath.

Table 1 below exemplifies the properties of the fluid management layers as specified in the inventive samples in comparison to the fluid management layers as specified in the comparative samples. More specifically, the inventive samples have a lower machine direction (MD)/cross direction (CD) peak load ratio and a lower MD/CD bending length flexural rigidity ratio. This gives the fluid management layer more flexibility in the MD and more resiliency in the CD. The flexibility in the MD provides a better and closer fit to the user's body, which creates a more comfortable experience and provides increased protection, less leaks. The increased CD resiliency allows the product to increase recovery, such as after the layer is compressed from, for example, the opening and closing of a user's thighs, and reduce bunching, such as when the user exercises or sleeps.

Additionally, the inventive samples achieve a higher caliper factor while using small fiber sizes. The high caliper factor makes the fluid management layer more cushiony and comfortable when used in a final product, such as a feminine hygiene pad. The high caliper factor also provides for more volume for fluid to distribute within the material, and the small fibers, less than 2 dtex, create a smaller pore size that sufficiently drains the topsheet despite the high caliper factor making the feminine hygiene pad feel dry and comfortable to the user and preventing leakage from having fluid remain on the topsheet (insufficient draining). By contrast, large fibers, such as the 10 dtex PET fibers in Comparative Sample 2, are used to increase caliper factor. The large fibers in Comparative Sample 2 result in an increase in pore size that drains the topsheet less efficiently than our Inventive Samples. For example, sample Comparative Sample 3 has similar sized fibers to our inventive samples but only achieves a caliper factor (mm per 10 gsm) of 0.10, whereas the Inventive Samples 1-5 have a larger caliper factor, from about 0.25 to about 0.3 with similarly sized fibers. However, due to the needlepunch processing and activation of the layer, a higher caliper with smaller fibers achieves improved fluid handling.

The, the Inventive Samples have improved fluid handling properties and improved user fit in comparison to the Comparative Samples.

TABLE 1

| | Inventive | Inventive | Inventive | Inventive | Inventive | Comparative | Comparative | Comparative |
|---|---|---|---|---|---|---|---|---|
| | Sample 1 | Sample 2 | Sample 3 | Sample 4 | Sample 5 | Sample 1 | Sample 2 | Sample 3 |
| Basis Weight | 71.68 | 65.61 | 64.98 | 57.54 | 62.00 | 55.89 | 65.4 | 58.5 |
| (g/m²) | | | | | | | | |
| Caliper 0.5 kPa | 1.85 | 1.88 | 1.88 | 1.79 | 1.87 | 0.72 | 0.99 | 0.65 |
| (mm) | | | | | | | | |
| Caliper Factor | 0.26 | 0.29 | 0.29 | 0.31 | 0.30 | 0.11 | 0.15 | 0.10 |
| (mm/10 gsm) | | | | | | | | |
| MD/CD Peak Load | 0.65 | 0.69 | 0.84 | 0.92 | 0.94 | 3.6 | 2.5 | 4.5 |
| ratio | | | | | | | | |
| CD Peak Load (N) | 45.61 | 7.17 | 9.85 | 7.20 | 36.70 | 8.98 | 17.28 | 16.7 |
| CD Elongation (%) | 100.86 | 33.56 | 41.50 | 47.95 | 104.50 | 139.78 | 115.9 | 135.6 |
| CD Bending length | 3.07 | 3.07 | 3.11 | 1.53 | — | — | 2.1 | 0.32 |
| flexural rigidity | | | | | | | | |
| (mN*cm) | | | | | | | | |
| MD Peak Load (N) | 29.46 | 4.98 | 8.27 | 6.59 | 34.40 | 32.04 | 43.6 | 75.9 |
| MD Elongation (%) | 100.86 | 59.76 | 67.43 | 57.32 | 65.60 | 40.36 | 38.71 | 32.1 |
| MD Bending length | 6.31 | 3.01 | 4.19 | 2.35 | — | — | 15.98 | 8.95 |
| flexural rigidity | | | | | | | | |
| (mN*cm) | | | | | | | | |
| MD/CD Bending | 2.1 | 1.0 | 1.3 | 1.5 | — | — | 7.6 | 28.0 |
| length flexural | | | | | | | | |
| rigidity ratio | | | | | | | | |
| Air Permeability | 203.93 | 150.87 | 147.60 | 221.00 | 99.50 | 184.67 | 211.67 | 159 |
| (m³/m²/min) | | | | | | | | |
| Pore size (μm) | — | 71.9 | 63.1 | 60.1 | 77.7 | — | 102.3 | — |

As previously mentioned, the Inventive Samples have undergone some activation during the manufacturing process. Activation refers to processing, such as by heating, the layer to form bonds between the bonding fibers, such as the bicomponent fibers of Inventive Samples 1-5. Various levels of activation may be used leading to different levels of bonding between the bonding fibers. Low activation including a percent bonded volume less than 2%, moderate activation includes a percent bonded volume greater than about 2% to about 4.5%, and high activation includes greater than about 4.5% or from about 4.5% to about 9%. The higher the activation, the higher the increase in tensile strength and bending length flexural rigidity. Generally, fluid management layers with low or no activation have low tensile strength that makes processing the layer into a finish feminine hygiene pad impossible or very difficult. By contrast, fluid management layers with high levels of activation become very stiff and make the product stiff and uncomfortable during use. Thus, a fluid management layer having a tensile strength, referred to as MD peak load, of greater than about 3 is able to be processed and a MD bending length flexural rigidity of from about 2 to about 60 so that the product comfortably fits the user. Table 2 below includes data on three Inventive Samples at various levels of activation, and Table 3 is a comparison of several of the Inventive Samples that have a similar formulation but have been activated at different levels.

Inventive Samples 1-5 are as previously described herein.

Inventive Sample 6 includes a fluid management layer having a basis weight of 66.17 gsm and having 20 percent by weight cellulose fibers having a 1.3 dtex; 50 percent by weight tri-lobal polypropylene fibers having a 1.0 dtex; and 30 percent by weight bi-component fibers having a 1.7 dtex. The cellulosic fibers are viscose fibers. The bi-component fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter. Inventive Sample 6 was activated by applying heat at an elevated temperature.

Inventive Sample 7 includes a fluid management layer having a basis weight of 66.17 gsm and having 25 percent by weight cellulose fibers having a 1.3 dtex; and 75 percent by weight bi-component fibers having a 1.7 dtex. The bi-component fibers have a polyethylene terephthalate component and polyethylene component in a core-sheath configuration, where the polyethylene is the sheath. The cellulose fibers are viscose fibers. The fluid management layer includes a plurality of vertical fiber bundles having a stitch density of 150 punches per square centimeter. The Inventive Sample 7 fluid management layer was highly activated and, as indicated above, includes a greater amount of bonding fibers, which are the bicomponent fibers, as compared with Inventive Samples 2-4, 6, and 8.

Inventive Sample 8 includes a fluid management layer having a basis weight of 68.99 gsm and having 25 percent by weight cellulose fibers having a 0.9 dtex; 45 percent by weight tri-lobal polypropylene fibers having a 1.0 dtex; and 30 percent by weight bi-component fibers having a 1.7 dtex. The cellulosic fibers are viscose fibers. The bi-component fibers have a first component polyethylene terephthalate and a second component polyethylene in a core-sheath configuration, where the polyethylene is the sheath. The fluid management layer includes a plurality of vertical fiber bundles having a stitch density of 150 punches per square centimeter. The Inventive Sample 8 fluid management layer was not activated.

TABLE 2

| | Inventive | Inventive | Inventive |
|---|---|---|---|
| | Sample 6 | Sample 7 | Sample 8 |
| Activation Level | High | Moderate | None |
| Basis Weight | 71.68 | 65.61 | 64.98 |
| (g/m²) | | | |
| Caliper 0.5 kPa | 1.85 | 1.88 | 1.88 |
| (mm) | | | |
| Caliper Factor | 0.26 | 0.29 | 0.29 |
| (mm/10 gsm) | | | |
| MD/CD Peak Load | 0.65 | 0.69 | 0.84 |
| ratio | | | |
| CD Peak Load (N) | 45.61 | 7.17 | 9.85 |
| CD Elongation (%) | 100.86 | 33.56 | 41.50 |
| CD Bending length | 3.07 | 3.07 | 3.11 |
| flexural rigidity | | | |
| (mN*cm) | | | |
| MD Peak Load (N) | 29.46 | 4.98 | 8.27 |
| MD Elongation (%) | 100.86 | 59.76 | 67.43 |
| MD Bending length | 6.31 | 3.01 | 4.19 |
| flexural rigidity | | | |
| (mN*cm) | | | |
| MD/CD Bending | 2.1 | 1.0 | 1.3 |
| length flexural | | | |
| rigidity ratio | | | |
| Air Permeability | 203.93 | 150.87 | 147.60 |
| (m³/m²/min) | | | |
| Pore size (μm) | — | 71.9 | 63.1 |

TABLE 3

| | Inventive | Inventive | Inventive | Inventive | Inventive | Inventive | Inventive | Inventive |
|---|---|---|---|---|---|---|---|---|
| | Sample 1 | Sample 7 | Sample 2 | Sample 3 | Sample 4 | Sample 5 | Sample 6 | Sample 8 |
| Activation | Moderate | High | Moderate | Moderate | Moderate | Moderate | High | None |
| CD Peak Load | 45.61 | 35.52 | 7.2 | 9.85 | 7.20 | 36.7 | 24.88 | 1.93 |
| (N/inch) | | | | | | | | |
| CD Bending | 3.07 | 21.6 | 3.07 | 3.11 | 1.53 | | 8.9 | 0.35 |
| Length - Flexural | | | | | | | | |
| Rigidity(mN*cm) | | | | | | | | |
| MD Peak Load | 29.46 | 36.35 | 5.0 | 8.27 | 6.59 | 34.4 | 23.66 | 2.28 |
| (N/inch) | | | | | | | | |
| MD Bending | 6.31 | 52.33 | 3.01 | 4.19 | 2.35 | | 17.98 | 0.17 |
| Length (mN*cm) | | | | | | | | |
| MD/CD Peak Load | 0.65 | 1.02 | 0.69 | 0.84 | 0.92 | 0.94 | 0.95 | 1.2 |
| Ratio | | | | | | | | |
| MD/CD Bending | 2.1 | 2.4 | 1.0 | 1.3 | 1.5 | | 2.0 | 0.5 |
| Length Ratio | | | | | | | | |
| Percent Bonded | | 7.3 | — | 3.9 | — | — | 4.9 | — |
| Volume (%) | | | | | | | | |

As shown in Table 2, as the level of activation decreases, both the MD peak load and the CD peak load decreases. Additionally, as the level of activation decreases, the bending length also decreases. The amount of increase or decrease is also dependent on the types of fibers used. For example, as shown in Table 3, Inventive Samples 1 and 7 each include a fluid management layer having about 75 weight percent of bonding fibers, which are bicomponent fibers. Inventive Sample 1 underwent moderate activation and Inventive Sample 7 underwent high activation. As shown, Inventive Sample 7 has both an MD peak load, MD bending length flexural rigidity, and CD bending length flexural rigidity that are considerably higher than Inventive Sample 1 that was moderately activated. Further, the percent bonded volume is greater in Inventive Sample 7 than Inventive Sample 1 which indicates that Inventive Sample 7 includes greater bonding, resulting in a stiffer fluid management layer.

Still referring to Table 3, Inventive Samples 2-6 and 8 each includes a three component fiber blend of similar formulation. As shown above, Inventive Samples 2-5 underwent moderate activation, Inventive Sample 6 underwent high activation and Inventive Sample 8 underwent no activation. As shown, Inventive Sample 6 has a CD peak load and a MD peak load that is greater than Inventive Sample 2-5, and both an MD and CD bending length flexural rigidity that is greater than Inventive Samples 2-5. Additionally, Inventive Sample 8 has a CD peak load and a MD peak load that is less than Inventive Sample 2-5, and both an MD and CD bending length flexural rigidity that is less than Inventive Samples 2-5. In summary, the inventors have found that based on the fiber formulation the amount of activation should be selected such that the fluid management layer is not too stiff, caused by a relatively higher MD and/or CD bending length flexural rigidity, or unable to be processed, caused by a relatively low MD and/or CD peak load.

Additionally, based on the level of activation and the blend of fibers, the Percent Bonded Volume (%), as determined by the % Bonded Volume Measurement via Micro-CT Method disclosed herein, the number of bonds between fibers can be increased with increasing activation. Alternatively or additionally, for those layers including a greater percentage of bonding fibers, which for the samples selected are bicomponent fibers, the greater the percentage of bonded volume of the layer. Generally, the greater the percentage of bonded volume the stronger the layer due to the increased number of bonds.

As previously discussed herein, not only does the inventive fluid management layer as discussed herein exhibit superior fluid handling properties and increased user comfort, but absorbent articles including this inventive fluid management layer exhibit improved fluid handling and increased user comfort. Table 4 below includes data on various inventive products including an inventive fluid management layer as specified herein.

Inventive Products 1, 2, 3, 4, and 5 all include the same topsheet. The topsheet is a nonwoven topsheet having a basis weight of 24 gsm and being an air-through bonded nonwoven. The air-through bonded nonwoven included bi-component fibers having a 4.4 dtex, where polyethylene terephthalate and polyethylene were in a core-sheath configuration, with polyethylene as the sheath. The topsheet included 60% hydrophilic fibers and 40% hydrophobic fibers by weight of the fibers.

Inventive Products 1, 2, 3, 4, and 5 all include the same backsheet. The backsheet is 12 gsm polypropylene film, available from RKW.

Inventive Products 1, 2, 3, and 4 all include the same core. The absorbent core is an airlaid absorbent core comprising pulp fibers, absorbent gelling material, and bico fibers, having a basis weight of 150 gsm available from Glatfelter, York, Pa., USA and having 22 gsm of absorbent gelling material. Inventive Product 5 includes an airlaid absorbent core comprising pulp fibers, absorbent gelling material, and bico fibers, having a basis weight of 150 gsm available from Glatfelter, York, Pa., USA and having 26 gsm of absorbent gelling material.

Inventive Product 1 additionally includes a secondary layer, also referred to as a fluid management layer, between the topsheet and core. The fluid management layer has a basis weight of 71.68 gsm and includes 25 percent by weight cellulose fibers, which are viscose fibers, having a 1.3 dtex; 75 percent by weight bi-component fibers having a 1.7 dtex and having a first component polyethylene terephthalate and polyethylene in a core-sheath configuration where the polyethylene is the sheath. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Inventive Product 2 additionally includes a secondary layer, also referred to as fluid management layer, between the topsheet and core, having a basis weight of 65.61 gsm. The fluid management layer having 25 percent by weight cellulose fibers, which are viscose fibers, having a 0.9 dtex; 45 percent by weight tri-lobal polypropylene fibers having a 1.0 dtex; 30 percent by weight bi-component fibers having a 1.7 dtex and having a first component polyethylene terephthalate and polyethylene in a core-sheath configuration where the polyethylene is the sheath. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Inventive Product 3 additionally includes a secondary layer, also referred to herein as a fluid management layer, between the topsheet and core, having a basis weight of 64.98 gsm. The fluid management layer having 25 percent by weight cellulose fibers, which are viscose fibers, having a 0.9 dtex; 45 percent by weight tri-lobal polypropylene fibers having a 1.0 dtex; and 30 percent by weight bi-component fibers having a 1.7 dtex and having a first component polyethylene terephthalate and polyethylene in a core-sheath configuration where the polyethylene is the sheath.

Inventive Product 4 additionally includes a secondary layer, also referred to herein as a fluid management layer, between the topsheet and core, having a basis weight of 65.61 gsm. The fluid management layer having 25 percent by weight cellulose fibers, which are viscose fibers, having a 1.3 dtex; and 75 percent by weight bi-component fibers having a 1.7 dtex and having a first component polyethylene terephthalate and polyethylene in a core-sheath configuration where the polyethylene is the sheath.

Inventive Product 5 additionally includes a secondary layer, also referred to herein as a fluid management layer, between the topsheet and core, having a basis weight of 71.68 gsm. The fluid management layer having 25 percent by weight cellulose fibers, which are viscose fibers, having a 1.3 dtex; and 75 percent by weight bi-component fibers having a 1.7 dtex and having a first component polyethylene terephthalate and polyethylene in a core-sheath configuration where the polyethylene is the sheath.

For Inventive Products 1-5, each of the fluid management layers includes a plurality of vertical fiber bundles having a stitch density of 150 punches per square centimeter.

TABLE 4

| | Inventive | Inventive | Inventive | Inventive | Inventive |
|---|---|---|---|---|---|
| | Product 1 | Product 2 | Product 3 | Product 4 | Product 5 |
| Dry MD 3 Point Bend | 2.9 | 2.8 | 2.8 | 2.7 | — |
| Caliper (mm) | | | | | |
| Z-Compression | 3.9 | 3.5 | 3.0 | 3.3 | — |
| Compression Energy | | | | | |
| (N*mm) | | | | | |
| Dry MD 3 Point Bend | 37.0 | 18.5 | 24.1 | 48.4 | — |
| Stiffness (N*m²) | | | | | |
| Wet Bunch Compression | 58 | 44 | 48 | 55 | — |
| % Recovery 1^{st} to 5^{th} cycle | | | | | |
| Wet Bunch Compression | 1.8 | 1.9 | 1.2 | 1.9 | — |
| Energy of Recovery 5th | | | | | |
| cycle (N*m²) | | | | | |
| RATR (3 ml gush) 1, 2, 3 | 6.7, 18.8, | 12.2, 40.9, | 13.5, 42.3, | — | 14.81, 42.93, |
| Acquisition Time (s) | 32.8 | 70.1 | 71.7 | | 83.36 |
| ATRM IFF (mg) | 55.0 | 26.4 | 38.8 | — | 39.9 |
| ATRM SFF (mg) | 23.2 | 13.2 | 34.3 | — | 336 |
| ATRM Rewet (mg) | 232.1 | 142.8 | 636.7 | — | 591 |

### Test Methods

### Pore Volume Distribution Test Method

The Pore Volume Distribution Test Method is used to determine the mean absorption pressure, mean desorption pressure and mean pore size of a porous test specimen by measuring the associated fluid movement into and out of said specimen as stepped, controlled differential pressure is applied to the specimen in a test sample chamber.

### Method Principle

For uniform cylindrical pores, the radius of a pore is related to the differential pressure required to fill or empty the pore by the equation:

$$Differential pressure = ( 2 γcosΘ ) / r ,$$

where γ=liquid surface tension, Θ=contact angle, and r=pore radius.

Pores contained in natural and manufactured porous materials are often thought of in terms such as voids, holes or conduits, and these pores are generally not perfectly cylindrical nor all uniform. One can nonetheless use the above equation to relate differential pressure to an effective pore radius, and by monitoring liquid movement into or out of the material as a function of differential pressure characterize the effective pore radius distribution in a porous material. (Because nonuniform pores are approximated as uniform by the use of an effective pore radius, this general methodology may not produce results precisely in agreement with measurements of void dimensions obtained by other methods such as microscopy.)

The Pore Volume Distribution Test Method uses the above principle and is reduced to practice using the apparatus and approach described in “Liquid Porosimetry: New Methodology and Applications” by B. Miller and I. Tyomkin published in The Journal of Colloid and Interface Science (1994), volume 162, pages 163-170, incorporated herein by reference. This method relies on measuring the increment of liquid volume that enters or leaves a porous material as the differential air pressure is changed between ambient (“lab”) air pressure and a slightly elevated air pressure (positive differential pressure) surrounding the specimen in a sample test chamber. The specimen is introduced to the sample chamber dry, and the sample chamber is controlled at a positive differential pressure (relative to the lab) sufficient to prevent fluid uptake into the specimen after the fluid bridge is opened. After opening the fluid bridge, the differential air pressure is decreased in steps to 0, and in this process subpopulations of pores acquire liquid according to their effective pore radius. After reaching a minimal differential pressure at which the mass of fluid within the specimen is at a maximum, differential pressure is increased stepwise again toward the starting pressure, and the liquid is drained from the specimen. It is during this latter draining sequence (from minimal differential pressure, or largest corresponding effective pore radius, to the largest differential pressure, or smallest corresponding effective pore radius), that the fluid retention by the sample (g/g) at each differential pressure is determined in this method. After correcting for any fluid movement for each particular pressure step measured on the chamber while empty, the fluid retention by the sample (g/g) for each pressure step is determined via dividing the equilibrium quantity of retained liquid (g) associated with this particular step by the dry weight of the sample (g).

### Sample Conditioning and Specimen Preparation

The Pore Volume Distribution Test Method is conducted on samples that have been conditioned in a room at a temperature of 23° C.±2.0° C. and a relative humidity of 50%+5%, all tests are conducted under the same environmental conditions and in such conditioned room. Any damaged product or samples that have defects such as wrinkles, tears, holes, and similar are not tested. Samples conditioned as described herein are considered dry samples for purposes of this invention. Three specimens are measured for any given material being tested, and the results from those three replicates are averaged to give the final reported values. Each of the three replicate specimens has a diameter of 50 mm. The dry mass of each prepared test specimen is recorded to the nearest 0.001 g.

### Apparatus

Apparatus suitable for this method is described in: “Liquid Porosimetry: New Methodology and Applications” by B. Miller and I. Tyomkin published in The Journal of Colloid and Interface Science (1994), volume 162, pages 163-170. Further, any pressure control scheme capable of controlling the sample chamber pressure between 0 mm H₂O and 1200 mm H₂O differential pressure may be used in place of the pressure-control subsystem described in this reference. One example of suitable overall instrumentation and software is the TRI/Autoporosimeter (Textile Research Institute (TRI)/Princeton Inc. of Princeton, N.J., U.S.A.). The TRI/Autoporosimeter is an automated computer-controlled instrument for measuring pore volume distributions in porous materials (e.g., the volumes of different size pores within the range from 1 to 1000 μm effective pore radii). Computer programs such as Automated Instrument Software Releases 2000.1 or 2003.1/2005.1 or 2006.2; or Data Treatment Software Release 2000.1 (available from TRI Princeton Inc.), and spreadsheet programs may be used to capture and analyze the measured data.

### Method Procedure

The wetting liquid used is a degassed hexadecane (CAS 544-76-3, reagent grade, available from any convenient source). Liquid density is 0.773 g/cm³, surface tension y to be 27+1 mN/m, and the contact angle cos Θ=1. A 90-mm diameter mixed-cellulose-ester filter membrane with a characteristic pore size of 1.2 μm (such Millipore Corporation of Bedford, MA, Catalogue #RAWP09025) is affixed to the porous frit (Monel plates with diameter of 90 mm, 6.4 mm thickness from Mott Corp., Farmington, CT, or equivalent) of the sample chamber.

Someone skilled in the art knows that it is critical to degas the test fluid as well as the frit/membrane/tubing system such that the system is free from air bubbles.

A metal weight weighing 414 g is placed on top of the sample to exert a constant confining pressure of 2.068 kPa during measurement.

The sequence of differential pressures that are run in the test, in mm H₂O, is as follows: 1100, 550, 367, 275, 220, 183, 138, 110, 92, 79, 69, 61, 55, 50, 46, 42, 39, 37, 34, 32, 31, 29, 28, 24, 22, 20, 18, 14, 9, 7, 6, 5, 4.5, 0, 4.5, 5, 6, 7, 9, 14, 18, 20, 22, 24, 28, 29, 31, 32, 34, 37, 39, 42, 46, 50, 55, 61, 69, 79, 92, 110, 138, 183, 220, 275, 367, 550, 1100.

The criterion for moving from one pressure step to the next is that fluid uptake/drainage from the specimen is measured to be less than 10 mg/min for 15 s.

A separate “blank” measurement is performed by following this method procedure on an empty sample chamber with no specimen or weight present on the membrane/frit assembly. Any fluid movement observed is recorded (g) at each of the pressure steps. Fluid retention data for a specimen are corrected for any fluid movement associated with the empty sample chamber by subtracting fluid retention values of this “blank” measurement from corresponding values in the measurement of the specimen.

### Determination of Mean Absorption Pressure, Mean Desorption Pressure

As described above, the capillary fluid taken up (g) by the test specimen during the filling cycle (absorption) for each pressure step of differential pressure is corrected for any effect of the empty chamber and then divided by the dry mass of the specimen to arrive at capillary fluid uptake normalized by the dry sample mass recorded to the nearest 0.001 g/g. Likewise, the capillary fluid retained (g) by the test specimen during its drainage cycle for each pressure step of differential pressure is corrected for any effect of the empty chamber and then divided by the dry mass of the specimen to arrive at capillary fluid drainage normalized by the dry mass recorded to the nearest 0.001 g/g. The test specimen is considered to be 100% saturated at the lowest differential pressure, and at this pressure step the normalized uptake is at its maximum. Percent saturation for each pressure step of differential pressure is calculated by dividing the normalized uptake at each pressure step by the maximum normalized uptake and then dividing by 100. For the filling cycle (absorption), the differential pressure value at 50% saturation is recorded as the Mean Absorption Pressure (MAP) to the nearest 0.01 cm H2O. For the drainage cycle, the differential pressure value at 50% saturation is recorded as the Mean Desorption Pressure (MDP) to the nearest 0.01 cm H2O.

The effective pore radius, R, at each pressure step of differential pressure is calculated using the following equation and recorded to the nearest 0.01 micron.

$$Differential {pressure}_{i} = ( 2 γcosΘ ) / {R}_{i}$$

where γ=liquid surface tension, Θ=contact angle, and r=pore radius.

The volume of fluid associated with each effective pore radius, V, is calculated using the following equation and the values are recorded to the nearest 0.01 mm³/g/micron.

$$V = \frac{\frac{( {w}_{i + 1} - {w}_{i} ) / d}{{R}_{i + 1} - {R}_{i}}}{{w}_{sample}}$$

where wᵢ is the fluid taken up (or retained, g), at pressure i (corrected for any effect of the empty chamber), d=fluid density (0.773 g/cm³), Rᵢ=pore radius at pressure i (microns) and wₛₐₘₚₗₑ=mass of the dry specimen (g).

In like fashion, repeat the entire procedure for a total of three replicate test specimens. The arithmetic mean of the three values for each recorded parameter (MAP, MDP) is reported.

### Acquisition Time and Rewet Method (ATRM)

This method describes how to measure gush acquisition time, interfacial free fluid amount as well as low and high pressure rewet values for an absorbent article loaded with new Artificial Menstrual Fluid (nAMF; preparation provided separately herein). A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low, and high pressure rewet are measured after the last liquid dose. Surface Free Fluid (SFF) is the amount of fluid that remains in the topshseet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2 C.° and 50%+2% relative humidity.

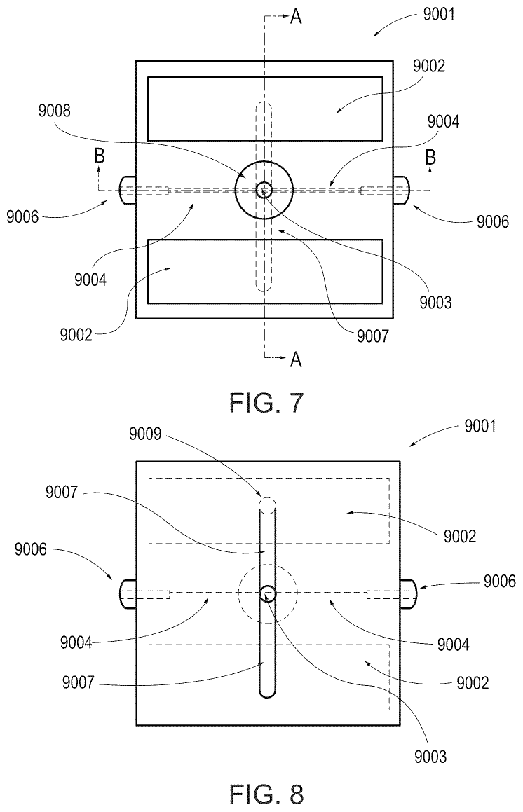

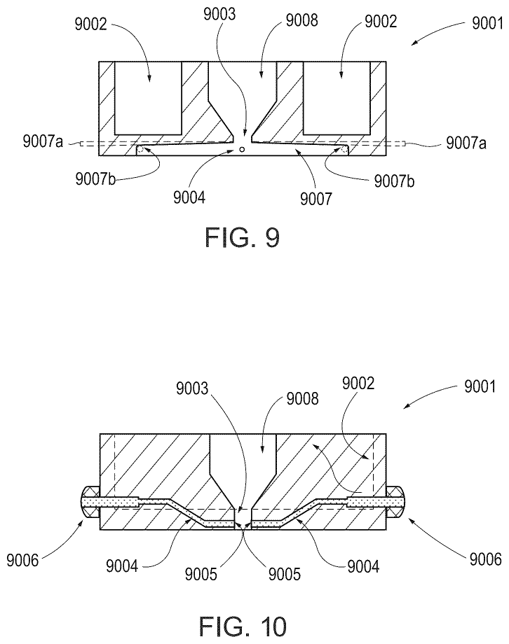

Referring to FIGS. 7 through 10, the strikethrough plate 9001 is constructed of Plexiglas, or equivalent, with an overall dimension of 10.2 cm long by 10.2 cm wide by 3.1 cm tall. A central, test fluid well 9008 has a circular opening of 25 mm in diameter is located at the top plane of the plate with initial lateral walls that extend 15 mm deep at a 90° angle and then slope downward at an angle of 82° for an additional depth of 7.5 mm to reach the test fluid reservoir 9003. The test fluid reservoir 9003 is concentric to the test fluid well 9008 and has a diameter of 6.6 mm with lateral walls that extend 5 mm deep at a 90° angle. The test fluid reservoir 9003 opens into the longitudinal fluid channel 9007 located at the bottom of the plate. The longitudinal fluid channel 9007 has lateral walls that initially extend 3.5 mm deep at the midpoint of the channel (just beneath the test fluid reservoir 9003), then slant downward at an angle 9007a of 0.72° towards each longitudinal end of the channel to a final depth of 3 mm. The longitudinal fluid channel opens to the bottom plane of the plate for the fluid to be introduced onto the underlying test sample. The longitudinal fluid channel 9007 is centered over the test fluid reservoir 9003 and extends in a direction that is perpendicular to the electrodes 9004. The longitudinal fluid channel 9007 has a width of 5 mm and a length of 80 mm, with lateral edges that are rounded with a radius 9007b of 1.0 mm. The longitudinal ends of the longitudinal fluid channel 9007 are rounded with a radius 9009 of 2.5 mm. Two wells 9002 (80.5 mm long by 24.5 mm wide by 25 mm deep) located outboard of the fluid reservoir, are filled with stainless steel shot (or equivalent) to adjust the overall mass of the plate to provide a constraining pressure of 0.10 psi (7.0 g/cm²) to the Test Area. The procedure for determining the test area is subsequently described herein. Electrodes 9004 are embedded in the plate 9001, connecting the exterior banana jacks 9006 to the inside wall 9005 of the longitudinal fluid channel 9003. A circuit interval timer is plugged into the jacks 9006, monitors the impedance between the two electrodes 9004, and measures the time from introduction of the nAMF into reservoir 9003 until the nAMF drains from the reservoir. The timer has a resolution of 0.01 sec.

A pretreatment plate is used in combination with a pretreatment weight to apply tiny droplets of nAMF to the surface of the test sample as a means to prime the surface of the test sample prior to the introduction of the full liquid dose. The pretreatment plate is constructed of Plexiglass, or equivalent, that is 14 inch (35.6 cm) long by 8 inch (20.3 cm) wide with a thickness of about 0.25 inch (6.4 mm). The pretreatment plate has five circular markers, each 5 mm in diameter, placed 1 cm apart (center to center) that are aligned along the longitudinal axis of the plate. The central marker is centered at the lateral midpoint of the plate. These markers indicate the placement of the nAMF droplets. The markers are located on the underside of the pretreatment plate and can be milled out or simply drawn on with a permanent marker, or equivalent. The pretreatment weight is 10.2 cm×10.2 cm and consists of a flat, smooth rigid material (e.g., stainless steel) with an optional handle. The pretreatment weight (including optional handle) has a total mass of 726 g+0.5 g to give a pressure of 0.10 psi (7.0 g/cm²) across the bottom surface area of the weight.

When measuring the interfacial fluid amounts, a rubber pad is used to provide a reproducibly flat surface that enables even pressure distribution. The IFF rubber pad is constructed from high strength neoprene rubber with 40 A durometer and a thickness of ⅛ inch (available from W.W. Grainger, Inc, item #1DUV4, or equivalent) and cut to dimensions of 6 inch (15.2 cm) by 6 inch (15.2 cm).

For the overall rewet portion of the test, a padded weight assembly that applies 0.5 psi (35.1 g/cm²) to the Test Area is required. The procedure for determining the test area is subsequently described herein. The rewet weight is constructed as follows. Lay a piece of polyethylene film (about 25 microns thick, any convenient source) horizontally flat on a rigid bench surface. A piece of polyurethane foam (25 mm thick, density of 1.0 lb/ft³, IDL 24 psi, available from Concord-Renn Co. Cincinnati, OH, or equivalent) is cut to 10.2 cm by 10.2 cm and then laid centered on top of the film. A piece of Plexiglas (10.2 cm by 10.2 cm and about 6.4 mm thick) is then stacked on top of the polyurethane foam. Next the polyethylene film is used to wrap the polyurethane foam and Plexiglas plate securing it with transparent tape. A metal weight with handle is stacked on top of, and fastened to, the Plexiglass plate such that the total mass of the padded weight assembly can be adjusted to apply a pressure of 0.5 psi (35.1 g/cm²) to the Test Area.

For the IFF, SFF and overall rewet steps, various layers of filter paper are required. The filter paper is conditioned at 23° C.±2° C. and 50%+2% relative humidity for at least 2 hours prior to testing. A suitable filter paper has a basis weight of about 88 gsm, a thickness of about 249 microns with an absorption rate of about 5 seconds and is available from Ahlstrom-Munksjo (Mt. Holly Springs, PA) as grade 632, or equivalent. The filter paper has dimensions of 5 inch by 5 inch (12.7 cm by 12.7 cm).

Test samples are conditioned at 23° C.±2° C. and 50%+2% relative humidity for at least 2 hours prior to testing. Test samples are removed from their outer packaging and the wrappers are opened to unfold the product, if applicable, using care not to press down or pull on the products while handling. No attempt is made to smooth out wrinkles. Tear the release paper between the wings, if applicable, and lay the sample on a horizontally flat, rigid surface with the body-side facing up (e.g., panty-side down). Determine the dose location as follows. For symmetrical products (i.e., the front of the product is the same shape and size as the back of the product when laterally divided along the midpoint of the longitudinal axis of the product), the dose location is the intersection of the midpoints of the longitudinal and lateral axes of the absorbent core. For asymmetrical products (i.e., the front of the product is not the same shape and size as the back of the product when laterally divided along the midpoint of the longitudinal axis of the product), the dose location is the midpoint of the product's wings at the lateral midpoint of the absorbent core. For products that have a foam core with holes and slits either punched out or printed, the dose location is the longitudinal midpoint of the hole-punched (or hole-printed) region at the lateral midpoint of the absorbent core. Once determined, mark the dose location with a small dot using a black, fine-tip, permanent marker. If wings are present, fold them to the back of the product.

Determine the Test Area of the test sample, as follows. This area will be used so that the mass of the strikethrough plate and the mass of the rewet weight can be properly adjusted to deliver the required pressure (0.1 psi and 0.5 psi, respectively). Measure the width of the absorbent core of the test sample as the distance between one lateral edge of the core to the other lateral edge of the core along a line that is positioned at the dosing location and runs perpendicular to the longitudinal axis of the test sample, and record as core width to the nearest 0.01 cm. Now multiply the core width by 10.2 cm (the length of the strikethrough plate and rewet weight) and record as Test Area to the nearest 0.1 cm². The total mass of the strikethrough plate is the Test Area multiplied by 7 g/cm². The total mass of the rewet weight is the Test Area multiplied by 35.1 g/cm².

The test sample is pretreated with nAMF as follows. Place the pretreatment plate onto a horizontally flat, rigid surface such that the side with the circular markers is facing down. Using a single channel, fixed volume pipettor, accurately dispense 50 μL of nAMF onto the topside of the pretreatment plate at the location of each of the five circular markers. Position the test sample above the pretreatment plate such that the body-side of the sample is facing the plate, the longitudinal axis of the sample and plate are aligned, and the pre-marked dose location on the test sample is centered over the central droplet of nAMF on the pretreatment plate. After properly positioned, place the test sample into contact with the pretreatment plate, then immediately apply the pretreatment weight onto the back side of the test sample, centering it over the dose location/central droplet of nAMF on the pretreatment plate. Start a 40 second timer. After 40 seconds have elapsed, remove the pretreatment weight from the test sample and remove the test sample from the pretreatment plate. Invert the test sample so that the body-side is facing up, place it onto a horizontally flat, rigid surface and immediately proceed with the steps that follow.

The first acquisition time (ACQ-1) is measured as follows. Connect the electronic circuit interval timer to the strikethrough plate 9001 and zero the timer. Position the strikethrough plate 9001 above the body-side of the test sample such that the long axis of the longitudinal fluid channel 9007 on the underside of the strikethrough plate 9001 is aligned with the longitudinal axis of the test sample, and ensure that the fluid reservoir 9003 is centered over the pre-marked dose location on the test sample. To note, nAMF should be visible through the fluid reservoir 9003 at the dose location on the test sample. After properly positioned, gently place the strikethrough plate 9001 onto the test sample. Using an adjustable volume pipettor, accurately dispense 2.0 mL of nAMF into the fluid well 9008 in the strikethrough plate 9001. The fluid is dispensed, without splashing, along the angled walls of the fluid well 9008 within a period of 3 seconds or less. Immediately after the fluid has been acquired, record the first acquisition time (ACQ-1) displayed on the circuit interval timer to the nearest 0.1 seconds. Leave the strikethrough plate 9001 in place on the test sample, and immediately start a 2 minute timer.

After 2 minutes have elapsed, measure the first Interfacial Free Fluid (IFF-1) as follows. Place the IFF rubber pad onto a horizontally flat, rigid surface. Measure the mass of one layer of filter paper to the nearest 0.0001 g and record as IFF-1ᵢₙᵢₜᵢₐₗ. Place the filter paper centered onto the IFF rubber pad. Transfer the strikethrough plate 9001 from the test sample to the pre-weighed filter paper such that the plate is centered on the filter paper, and immediately start an 8 minute timer. After 10 seconds have elapsed on the 8 minute timer, remove the strikethrough plate from the filter paper and gently replace it back onto the test sample, exactly as previously positioned. Within the next 10 seconds, measure the mass of the filter paper to the nearest 0.0001 g and record as IFF-1_{final}.

The second acquisition time (ACQ-2) is measured as follows. After 8 minutes have elapsed, apply the second gush of fluid using an adjustable volume pipettor to accurately dispense 4.0 mL of nAMF into the fluid well 9008 in the strikethrough plate 9001, as previously described. Immediately after the fluid has been acquired, record the second acquisition time (ACQ-2) displayed on the circuit interval timer to the nearest 0.1 second. Leave the strikethrough plate 9001 in place on the test sample, and immediately start a 2 minute timer.

After 2 minutes have elapsed, measure the second Interfacial Free Fluid (IFF-2) as follows. Place the IFF rubber pad onto a horizontally flat, rigid surface. Measure the mass of a fresh, single layer of filter paper to the nearest 0.0001 g and record as IFF-2ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and gently replace it back onto the test sample, exactly as previously positioned. Within the next 10 seconds, measure the mass of the filter paper to the nearest 0.0001 g and record as IFF-2_{final}.

The third acquisition time (ACQ-3) is measured as follows. After 8 minutes have elapsed, apply the third gush of fluid using an adjustable volume pipettor to accurately dispense 2.0 mL of nAMF into the fluid well 9008 in the strikethrough plate 9001, as previously described. Immediately after the fluid has been acquired, record the third acquisition time (ACQ-3) displayed on the circuit interval timer to the nearest 0.1 second. Leave the strikethrough plate 9001 in place on the test sample, and immediately start a 2 minute timer.

After 2 minutes have elapsed, measure the third Interfacial Free Fluid (IFF-3) as follows. Place the IFF rubber pad onto a horizontally flat, rigid surface. Measure the mass of a fresh, single layer of filter paper to the nearest 0.0001 g and record as IFF-3ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and set it on its side so that the pad-side of the plate is not contacting the bench. Within the next 10 seconds, measure the mass of the filter paper to the nearest 0.0001 g and record as IFF-3_{final}.

Measure Surface Free Fluid (SFF) as follows. After 8 minutes have elapsed, measure the mass of a fresh stack of 5 filter papers to the nearest 0.0001 g and record as SFFᵢₙᵢₜᵢₐₗ. Place the stack of filter papers on top of the body-side of the test sample such that they are centered over the dose location. Now gently place the strikethrough plate 9001 on top of the filter papers such that the pad-side of the plate is centered on the filter papers, and immediately start a 10 second timer. After 10 seconds have elapsed, remove the strikethrough plate 9001 from the filter papers and set it aside. Measure the mass of the stack of 5 filter papers to the nearest 0.0001 g and record as SFF_{final}. Immediately proceed to the next step.

Measure overall rewet as follows. Measure the mass of a fresh stack of 5 filter papers to the nearest 0.0001 g and record as REWETᵢₙᵢₜᵢₐₗ. Place the filter papers on top of the body-side of the test sample such that they are centered over the dose location. Now place the padded Rewet Weight on top of the stack of filter papers such that the weight is centered on the filter paper stack, and immediately start a 30 second timer. After 30 seconds have elapsed, remove the rewet weight and measure the mass of the stack of 5 filter papers to the nearest 0.0001 g, then record as REWET_{final}. Discard the sample and thoroughly clean and then dry the fluid well 9008, fluid reservoir 9003, longitudinal fluid channel 9007 and the bottom surface of the strikethrough plate 9001 prior to testing the next sample.

Make the following calculations for each of the parameters measured, as follows. Calculate Total Gush Absorbency Time as the sum of ACQ-1, ACQ-2 and ACQ-3, and record to the nearest 0.1 second. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate Total IFF as the sum of IFF-1, IFF-2 and IFF-3, and record to the nearest 0.1 g. Calculate SFF by subtracting SFFᵢₙᵢₜᵢₐₗ from SFF_{final}, and record to the nearest 0.0001 g. Calculate Total IFF+SFF as the sum of Total IFF and SFF, and record to the nearest 0.1 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the arithmetic mean of the three individually recorded measurements for each Acquisition Time (ACQ-1, ACQ-2 and ACQ-3) to the nearest 0.1 seconds, Total Gush Absorbency Time to the nearest 0.1 seconds, Interfacial Free Fluid (IFF-1, IFF-2 and IFF-3) to the nearest 0.0001 g, Total IFF to the nearest 0.1 g, Surface Free Fluid (SFF) to the nearest 0.0001 g, Total IFF+SFF to the nearest 0.1 g, and Overall Rewet to the nearest 0.0001 g.

### Fiber Decitex (Dtex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%+2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10000 m \times {a}_{k} \times {ρ}_{k} \times {10}^{- 6}$$

where dₖ is in units of grams (per calculated 10,000 meter length), aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

The denier value for each fiber type in the web material is calculated as follows:

$$denier = 0.9 \times {d}_{k}$$

where dₖ is in units of grams (per calculated 10,000 meter length) and denier is in units of grams. Denier is reported to the nearest 0.1 g along with the fiber type (e.g., PP, PET, cellulose, PP/PET bico).

### Basis Weight

The basis weight of a test sample is the mass (in grams) per unit area (in square meters) of a single layer of material and is measured in accordance with compendial method WSP 130.1. The mass of the test sample is cut to a known area, and the mass of the sample is determined using an analytical balance accurate to 0.0001 grams. All measurements are performed in a laboratory maintained at 23° C.±2° C. and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Air Permeability

The measurements for air permeability provided herein were obtained by using Worldwide Strategic Partners (WSP) Test Method 70.1.

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm; however, a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it is larger than the pressure foot.

To measure caliper, first zero the micrometer against the horizontal flat reference platform. Place the test specimen on the platform with the test location centered below the pressure foot. Gently lower the pressure foot with a descent rate of 3.0 mm+1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously, is the caliper (mm) per 10 gsm of basis weight of the sample. So, the equation is caliper/(basis weight/10).

### Density

Density is calculated based upon the basis weight and caliper with appropriate unit conversion to arrive at g/cc.

### Material Compositional Analysis

The quantitative chemical composition of a test specimen comprising a mixture of fiber types is determined using ISO 1833-1. All measurements are performed in a laboratory maintained at 23° C.±2° C. and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Compression Caliper

The caliper, or thickness, of a test specimen is measured as the distance between a reference platform on which the specimen rests and a pressure foot that exerts a specified amount of pressure onto the specimen over a specified amount of time. For this method, a series of pressures are applied to the test specimen for a specified time, with a recovery period in between. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure onto the test specimen at each of the specified pressures (+0.01 kPa) in the stepped pressure series 0.50, 1.00, 2.00, 3.00, 5.00, and 0.50 kPa. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has an area of 25 cm², however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen from a sample of the material being evaluated. The test specimen is obtained from an area free of folds or wrinkles, and it is larger than the pressure foot.

To measure caliper, first ensure the pressure to be exerted onto the sample is adjusted to the first pressure in the stepped pressure series, 0.50 kPa. Now zero the micrometer against the horizontal flat reference platform. Place the test specimen on the platform with the test location centered below the pressure foot. Gently lower the pressure foot with a descent rate of 3.0 mm+1.0 mm per second until the full pressure is exerted onto the test specimen. Wait 4 seconds, then record the caliper of the test specimen to the nearest 0.001 mm noting the test pressure applied. Remove the pressure from the test specimen and set a 30 second timer accurate to 0.1 seconds (any convenient source). Now adjust the pressure to the next pressure setting in the stepped series, 1.00 kPa, and zero the micrometer against the horizontal flat reference platform. After 30 seconds have elapsed, place the test specimen onto the platform with the same test location centered below the pressure foot. In like fashion, lower the pressure foot and record the caliper of the test specimen to the nearest 0.001 mm noting the pressure applied. Remove the pressure from the test specimen and set a 30 second timer. Repeat this entire process for each of the pressures in the stepped pressure series, in the order as follows: 0.50, 1.00, 2.00, 3.00, 5.00, 0.50 kPa. Record the caliper to the nearest 0.001 mm for each pressure, noting the pressure applied. For the 0.50 kPa pressure setting, note 0.50 kPa Initial and 0.50 kPa Final, respectively, along with the caliper. The caliper foot is to be applied to the same test location on the test specimen for each pressure applied.

In like fashion, repeat for a total of three replicate test specimens. Calculate the arithmetic mean for the caliper measurements at each pressure, and report as Caliper to the nearest 0.001 mm, noting the pressure applied for each, along with Initial and Final for the 0.50 kPa setting. From these results, Caliper Decrease can be calculated between any of the pressure settings used by simply subtracting the caliper obtained at a higher pressure from the caliper obtained at a lower pressure, and reporting to the nearest 0.001 mm.

### Repetitive Acquisition Time and Rewet (RATR)

Acquisition time is measured for an absorbent article dosed with Artificial Menstrual Fluid (AMF) as described herein, using a strikethrough plate and an electronic circuit interval timer. The time required for the absorbent article to acquire a series of doses of AMF is recorded. Subsequent to the acquisition test, a rewet test is performed. All measurements are performed in a laboratory maintained at 23° C.±2° C. and 50%±2% relative humidity.

Referring to FIGS. 7-10, the strikethrough plate 9001 is constructed of Plexiglas with an overall dimension of 10.2 cm long by 10.2 cm wide by 3.2 cm tall. A longitudinal channel 9007 that runs the length of the plate is 13 mm deep, 28 mm wide at the top plane of the plate, with lateral walls that slope downward at 65° to a 15 mm wide base. A central test fluid well 9009 is 26 mm long, 24 mm deep, 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying test sample. The test fluid reservoir 9003 has an overall length of 25 mm, width of 15 mm, and depth of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 6 mm apart. The lateral sides of the reservoir bow outward at a radius 9012 of 14 mm bounded by the overall width 2013 of 15 mm. Two wells 9002 (80.5 mm long×24.5 mm wide×25 mm deep) located outboard of the lateral channel, are filled with lead shot (or equivalent) to adjust the overall mass of the plate to provide a constraining pressure of 0.25 psi (17.6 g/cm²) to the test area. Electrodes 9004 are embedded in the plate 9001, connecting the exterior banana jacks 9006 to the inside wall 9005 of the fluid reservoir 9003. A circuit interval timer is plugged into the jacks 9006, and monitors the impedance between the two electrodes 9004, and measures the time from introduction of the AMF into reservoir 9003 until the AMF drains from the reservoir. The timer has a resolution of 0.01 sec.

For the rewet portion of the test, the pressure applied to the test sample is 1.0 psi. The rewet weight is constructed such that the dimensions of the bottom face of the weight match the dimensions of the strikethrough plate, and the total mass required is calculated to give a pressure of 1.0 psi over the bottom face of the weight. Thus, the bottom face of the weight is 10.2 cm long by 10.2 cm wide, and constructed of a flat, smooth rigid material (e.g., stainless steel) to give a mass of 7.31 kg.

For each test sample, seven plies of filter paper cut to 150 mm diameter are used as the rewet substrate. The filter paper is conditioned at 23° C.±2° C. and 50%±2% relative humidity for at least 2 hours prior to testing. A suitable filter paper has a basis weight of about 74 gsm, a thickness of about 157 microns with medium porosity, and is available from VWR International as grade 413.

Test samples are removed from all packaging using care not to press down or pull on the products while handling. No attempt is made to smooth out wrinkles. The test samples are conditioned at 23° C.±2° C. and 50%±2% relative humidity for at least 2 hours prior to testing. Determine the dose location as follows. For symmetrical samples (i.e., the front of the sample is the same shape and size as the back of the sample when laterally divided along the midpoint of the longitudinal axis of the sample), the dose location is the intersection of the midpoints of the longitudinal axis and lateral axis of the sample. For asymmetrical samples (i.e., the front of the sample is not the same shape and size as the back of the sample when laterally divided along the midpoint of the longitudinal axis of the sample), the dose location is the intersection of the midpoint of the longitudinal axis of the sample and a lateral axis positioned at the midpoint of the sample's wings.

The required mass of the strikethrough plate must be calculated for the specific dimensions of the test sample such that a constraining pressure of 0.25 psi is applied. Measure and record the lateral width of the core at the dose location to the nearest 0.1 cm. The required mass of the strikethrough plate is calculated as the core width multiplied by the length of the strikethrough plate (10.2 cm) multiplied by 17.6 g/cm² and recorded to the nearest 0.1 g. Add lead shot (or equivalent) to the wells 9002 in the strikethrough plate to achieve the calculated mass.

Connect the electronic circuit interval timer to the strikethrough plate 9001 and zero the timer. Place the test sample onto a flat, horizontal surface with the body side facing up. Gently place the strikethrough plate 9001 onto the center of the test sample ensuring that the “H” shaped reservoir 9003 is centered over the predetermined dose location.

Using a mechanical pipette, accurately pipette 3.00 mL+0.05 mL of AMF into the test fluid reservoir 9003. The fluid is dispensed, without splashing, along the molded lip of the bottom of the reservoir 9003 within a period of 3 seconds or less. Immediately after the fluid has been acquired, record the acquisition time to the nearest 0.01 seconds and start a 5 minute timer. In like fashion, apply a second and third dose of AMF into the test fluid reservoir, with a 5 minute wait time between each dose. Record the acquisition times to the nearest 0.001 seconds. Immediately after the 3^{rd} dose of AMF has been acquired, start a 5 minute timer and prepare the filter papers for the rewet portion of the test.

Obtain the mass of 7 plies of the filter paper and record as Dry Massip to the nearest 0.001 grams. When 5 minutes have elapsed after the third acquisition, gently remove the strikethrough plate from the test sample and set aside. Place the 7 plies of pre-weighed filter paper onto the test sample, centering the stack over the dosing location. Now place the rewet weight centered over the top of the filter papers and start a 15 second timer. As soon as 15 seconds have elapsed, gently remove the rewet weight and set aside. Obtain the mass of the 7 plies of filter paper and record as Wet Mass_{fp} to the nearest 0.001 grams. Subtract the Dry Massip from the Wet Massip and report as Rewet Value to the nearest 0.001 grams. Thoroughly clean the electrodes 9004 and wipe off any residual test fluid from the bottom faces of the strikethrough plate and rewet weight prior to testing the next sample.

Immediately following the rewet portion of the test, proceed to the Stain Size method using the dosed test sample, as described herein.

In like fashion, repeat the entire procedure on ten replicate samples. The reported value is the arithmetic mean of the ten individual recorded measurements for Acquisition Times (first, second and third) to the nearest 0.001 seconds and Rewet Value to the nearest 0.001 grams.

### Stain Size Measurement Method

This method describes how to measure the size of a fluid stain visible on an absorbent article. This procedure is performed on test samples immediately after they have been dosed with test liquid according to a separate method, as described herein (e.g., the Repetitive Acquisition and Rewet method). The resultant test samples are photographed under controlled conditions. Each photographic image is then analyzed using image analysis software to obtain measurements of the size of the resulting visible stain. All measurements are performed at constant temperature (23° C.±2° C.) and relative humidity (50%±2%).

The test sample along with a calibrated ruler (traceable to NIST or equivalent) are laid horizontally flat on a matte black background inside a light box that provides stable uniform lighting evenly across the entire base of the light box. A suitable light box is the Sanoto MK50 (Sanoto, Guangdong, China), or equivalent, which provides an illumination of 5500 LUX at a color temperature of 5500K. A Digital Single-Lens Reflex (DSLR) camera with manual setting controls (e.g., a Nikon D40X available from Nikon Inc., Tokyo, Japan, or equivalent) is mounted directly above an opening in the top of the light box so that the entire article and ruler are visible within the camera's field of view.

Using a standard 18% gray card (e.g., Munsell 18% Reflectance (Gray) Neutral Patch/Kodak Gray Card R-27, available from X-Rite; Grand Rapids, MI, or equivalent) the camera's white balance is custom set for the lighting conditions inside the light box. The camera's manual settings are set so that the image is properly exposed such that there is no signal clipping in any of the color channels. Suitable settings might be an aperture setting of f/11, an ISO setting of 400, and a shutter speed setting of 1/400 sec. At a focal length of 35 mm the camera is mounted approximately 14 inches above the article. The image is properly focused, captured, and saved as a JPEG file. The resulting image must contain the entire test sample and distance scale at a minimum resolution of 15 pixels/mm.

To analyze the image, transfer it onto a computer running an image analysis software (a suitable software is MATLAB, available from The Mathworks, Inc, Natick, MA, or equivalent). The image resolution is calibrated using the calibrated distance scale in the image to determine the number of pixels per millimeter. The image is analyzed by manually drawing the region of interest (ROI) boundary around the visibly discernable perimeter of the stain created by the previously dosed test liquid. The area of the ROI is calculated and reported as the Overall Stain Area to the nearest 0.01 mm² along with notation as to which method was used to generate the test sample being analyzed (e.g., Repetitive Acquisition and Rewet).

This entire procedure is repeated on all of the replicate test samples generated from the dosing method(s). The reported value is the average of the individual recorded measurements for the Overall Stain Area to the nearest 0.01 mm² along with notation as to which method was used to generate the test samples that were analyzed (e.g., Repetitive Acquisition and Rewet).

### Artificial Menstrual Fluid (AMF) Preparation

The Artificial Menstrual Fluid (AMF) is composed of a mixture of defibrinated sheep blood, a phosphate buffered saline solution and a mucous component. The AMF is prepared such that it has a viscosity between 7.15 to 8.65 centistokes at 23° C.

Viscosity of the AMF is performed using a low viscosity rotary viscometer (a suitable instrument is the Cannon LV-2020 Rotary Viscometer with UL adapter, Cannon Instrument Co., State College, PA, or equivalent). The appropriate size spindle for the viscosity range is selected, and instrument is operated and calibrated as per the manufacturer. Measurements are taken at 23° C.±1° C. and at 60 rpm. Results are reported to the nearest 0.01 centistokes. Reagents needed for the AMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, available from Sterilized American Laboratories, Inc., Omaha, NE, or equivalent), 10% v/v lactic acid aqueous solution, 10% w/v potassium hydroxide aqueous solution, sodium phosphate dibasic anhydrous (reagent grade), sodium chloride (reagent grade), sodium phosphate monobasic monohydrate (reagent grade) and deionized water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add deionized water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add deionized water to volume. Mix thoroughly. To prepare the phosphate buffered saline solution, add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

The mucous component is a mixture of the phosphate buffered saline solution, potassium hydroxide aqueous solution, gastric mucin, and lactic acid aqueous solution. The amount of gastric mucin added to the mucous component directly affects the final viscosity of the prepared AMF. To determine the amount of gastric mucin needed to achieve AMF within the target viscosity range (7.15-8.65 centistokes at 23° C.) prepare 3 batches of AMF with varying amounts of gastric mucin in the mucous component, and then interpolate the exact amount needed from a concentration versus viscosity curve with a least squares linear fit through the three points. A successful range of gastric mucin is usually between 38 to 50 grams.

To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5° C. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range. After the 2.5 hours has elapsed, remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1° C.

Allow the temperature of the sheep blood and mucous component to come to 23° C.±1° C. Using a 500 mL graduated cylinder, measure the volume of the entire batch of the previously prepared mucous component and add it to a 1200 mL beaker. Add an equal volume of sheep blood to the beaker and mix thoroughly. Using the viscosity method previously described, ensure the viscosity of the AMF is between 7.15-8.65 centistokes. If not, the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### Dry MD Standard 3 Point Bend

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry test specimens. The intention of this method is to mimic deformation created in the x-y plane by a wearer of an absorbent article during normal use. All testing is performed in a room controlled at 23° C.±3° C. and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be move horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place the bar of the top fixture is parallel to and aligned front to back with the bars of the bottom fixture and is centered between the bars if the bottom fixture. Both fixtures include an integral adapter appropriate to fit the respective position on the universal test frame and lock into position such that the bars are orthogonal to the motion of the crossbeam of the test frame.

The gap (“span”) between the bars of the lower fixture is set to 25 mm+0.5 mm (center of bar to center of bar) with the upper bar centered at the midpoint between the lower bars. Set the gage (bottom of top bar to top of lower bars) to 1.0 cm.

The thickness (“caliper”) of the test specimen is measured using a manually-operated micrometer equipped with a pressure foot capable of exerting a steady pressure of 0.1 psi+0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3+1 mm/s until the full weight of the pressure is exerted onto the specimen. After 5 seconds elapse, the thickness is recorded as caliper to the nearest 0.01 mm.

The absorbent article samples are conditioned at 23° C.±3° C. and 50%±2% relative humidity two hours prior to testing. Dry test specimens are taken from an area of the sample that is free from any seams and residua of folds or wrinkles, and ideally from the center of pad (intersection of longitudinal and lateral midlines). The dry specimens are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, and marking the body-facing surface. The thickness of the test specimen is measured, as described herein, and recorded as dry specimen caliper to the nearest 0.01 mm. The mass of the test specimen is obtained and recorded as dry mass to the nearest 0.001 grams. The basis weight of the test specimen is calculated by dividing the mass (g) by the area (0.002581 m²) and recorded as dry specimen basis weight to the nearest 0.01 g/m². The bulk density of the test specimen is calculated by dividing the specimen basis weight (g/m²) by the specimen thickness (mm), then dividing the quotient by 1000, and recorded as dry specimen density to the nearest 0.01 g/cm³. In like fashion, five replicate dry test specimens are prepared.

The universal test frame is programed for a flexural bend test with the following settings. The motion of the crosshead is initiated such that the top fixture moves down with respect to the lower fixture at a rate of 1.0 mm/sec until the upper bar touches the top surface of the specimen with a nominal force of 0.02 N, then the crosshead continues to move down for an additional 12 mm. The crosshead is then immediately returned to the original gage at a rate of 1.0 mm/s. Force (N) and displacement (mm) data are continuously collected at 100 Hz throughout the test.

A dry test specimen is loaded onto the fixture such that it spans the two lower bars and is centered beneath the upper bar, with its sides parallel to the bars. For MD bending, the MD direction of the test specimen is perpendicular to the length of the 3 bars. The test is started and force and displacement data are continuously collected for the duration of the test.

A graph of force (N) versus displacement (mm) is constructed. From the graph, the maximum peak force is determined and recorded as dry MD peak load to the nearest 0.01 N. The maximum slope of the curve between the initial force and maximum force (during the loading portion of the curve) is calculated and recorded to the nearest 0.1 unit. The modulus is calculated using the following equation and recorded as dry MD modulus to the nearest 0.001 N/mm².

$$Modulus ( N / {mm}^{2} ) = ( Slope \times ( {Span}^{3} ) ) / ( 4 \times specimen width \times ( specimen {caliper}^{3} ) )$$

Bending stiffness is calculated as using the following equation and recorded as dry MD bending stiffness to the nearest 0.1 N mm².

$$Bending Stiffness ( N {mm}^{2} ) = Modulus \times Moment of Inertia$$

$$where Moment of Inertia ( {mm}^{4} ) = ( specimen width \times ( specimen {caliper}^{3} ) ) / 12$$

In like fashion, the procedure is repeated for all five replicates of the dry test specimens. The arithmetic mean among the five replicate dry test specimens is calculated for each of the parameters and reported as Dry Specimen Caliper to the nearest 0.01 mm, Dry Specimen Basis Weight to the nearest 0.01 g/m², Dry Specimen Density to the nearest 0.001 g/cm³, Dry MD Peak Load to the nearest 0.01 N, Dry MD Modulus to the nearest 0.001 N/mm², and Dry MD Bending Stiffness to the nearest N mm².

### Wet Bunch Compression Test

The Wet Bunch Compression test method measures the force versus displacement behavior across five cycles of load application (“compression”) and load removal (“recovery”) of a wetted absorbent article test sample that has been intentionally “bunched”, using a universal constant rate of extension test frame (a suitable instrument is the MTS Alliance using TestSuite software, as available from MTS Systems Corp., Eden Prairie, MN, or equivalent) equipped with a load cell for which the forces measured are within 1% to 99% of the limit of the cell. The test is executed on test specimens that are dosed with a specified amount of test fluid, as described herein. The intention of this method is to mimic the deformation created in the z-plane of the crotch region of an absorbent article, or components thereof, as it is worn by the wearer during sit-stand movements. All testing is performed in a room controlled at 23° C.±3° C. and 50%±2% relative humidity.

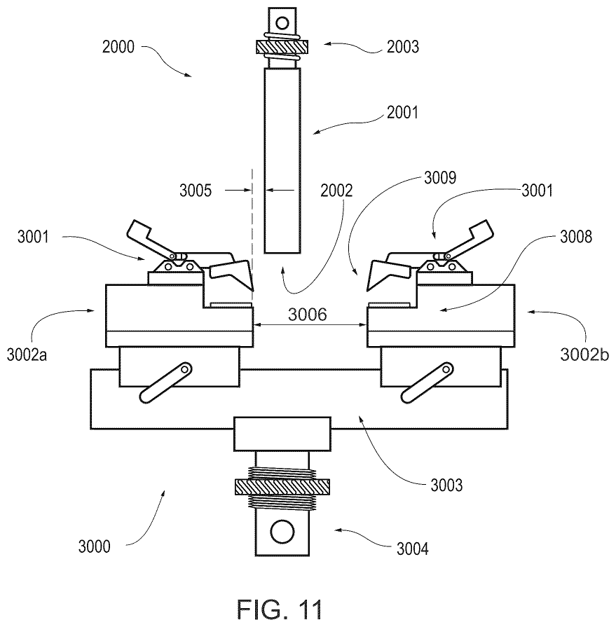

The test apparatus is depicted in FIGS. 11-13. The bottom stationary fixture 3000 consists of two matching sample clamps 3001 each 100 mm wide, each mounted on its own movable platform 3002a, 3002b. The clamp has a “knife edge” 3009 that is 110 mm long, which clamps against a 1 mm thick hard rubber face 3008. When closed, the clamps are flush with the interior side of its respective platform. The clamps are aligned such that they hold an un-bunched specimen horizontal and orthogonal to the pull axis of the tensile tester. The platforms are mounted on a rail 3003 which allows them to be moved horizontally left to right and locked into position. The rail has an adapter 3004 compatible with the mount of the tensile tester capable of securing the platform horizontally and orthogonal to the pull axis of the tensile tester. The upper fixture 2000 is a cylindrical plunger 2001 having an overall length of 70 mm with a diameter of 25.0 mm. The contact surface 2002 is flat with no curvature. The plunger 2001 has an adapter 2003 compatible with the mount on the load cell capable of securing the plunger orthogonal to the pull axis of the tensile tester.

Test samples are conditioned at 23° C.±3° C. and 50%±2% relative humidity for at least 2 hours before testing. Prepare the test specimen as follows. When testing an intact absorbent article, remove the release paper from any panty fastening adhesive on the garment facing side of the article, if present. Lightly apply talc powder to the adhesive to mitigate any tackiness. If there are cuffs, excise them with scissors so as not to disturb the topsheet or any other underlying layers of the article. Place the article, body facing surface up, on a benchtop. On the article, mark the intersection of the longitudinal midline and the lateral midline. Using a rectangular cutting die or equivalent cutting means, cut a specimen 100 mm in the longitudinal direction by 80 mm in the lateral direction, centered at the intersection of the midlines. When testing a material layer or layered components from an absorbent article, place the material layer or layered components on a benchtop and orient as it would be integrated into a finished article, i.e., identify the body facing surface and the lateral and longitudinal axis. Using a rectangular cutting die, or equivalent cutting means, cut a specimen 100 mm in the longitudinal direction by 80 mm in the lateral direction, centered at the intersection of the midlines. Measure the mass of the specimen and record to the nearest 0.001 grams. Calculate the basis weight of the specimen by dividing the mass (g) by the area (0.008 m²) and record as basis weight to the nearest 1 g/m². The test specimen is further prepared by applying a single dose of 10% w/v saline solution, as follows. The saline solution is prepared by adding 100 g of reagent grade NaCl to a 1 L volumetric flask and adding distilled water to the fill line. The volume of the liquid dose applied to the test specimen is 7 mL. The liquid dose is added using a calibrated Eppendorf-type pipettor, spreading the fluid over the complete body facing surface of the specimen within a period of approximately 3 sec. The wet specimen is tested 10.0 min±0.1 min after the dose is applied.

Program the tensile tester to zero the load cell, then lower the upper fixture at 2.00 mm/sec until the contact surface of the plunger touches the specimen and 0.02 N is read at the load cell. Zero the crosshead. Program the system to lower the crosshead 15.00 mm at 2.00 mm/sec then immediately raise the crosshead 15.00 mm at 2.00 mm/sec. This cycle is repeated for a total of five cycles, with no delay between cycles. Data is collected at 50 Hz during all compression/decompression cycles.

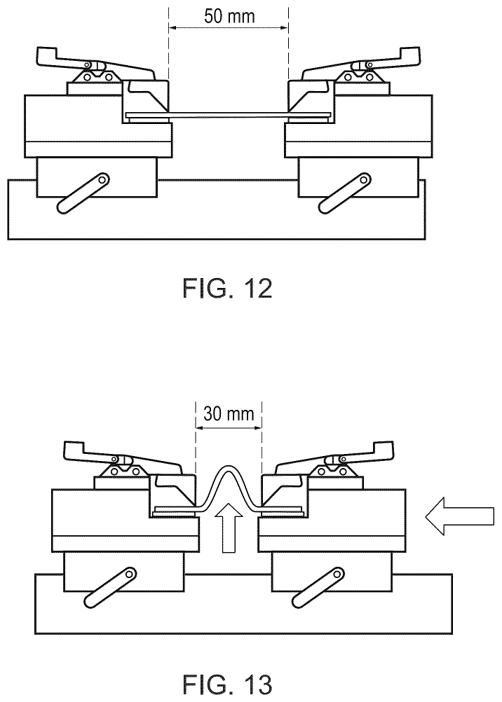

Position the left platform 3002a 2.5 mm from the side of the upper plunger (distance 3005). Lock the left platform into place. This platform 3002a will remain stationary throughout the experiment. Align the right platform 3002b 50.0 mm from the stationary clamp (distance 3006). Raise the upper probe 2001 such that it will not interfere with loading the specimen. Open both clamps 3001. Referring to FIG. 12, place the dry specimen with its longitudinal edges (i.e., the 100 mm long edges) within the clamps. With the dry specimen laterally centered, securely fasten both edges in the clamps. Referring to FIG. 13, move the right platform 3002b toward the stationary platform 3002a a distance 30.0 mm. Allow the dry specimen to bow upward as the movable platform is positioned. Now manually lower the probe 2001 until the bottom surface is approximately 1 cm above the top of the bowed specimen.

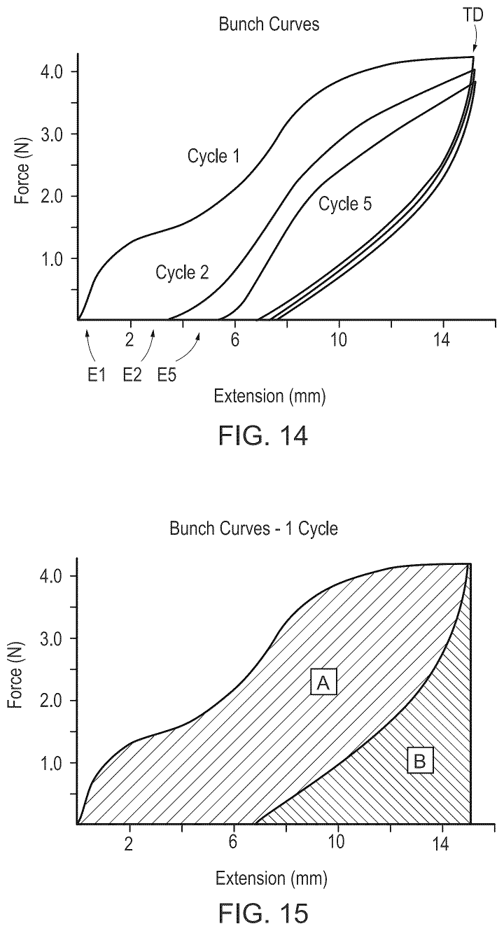

Start the test and continuously collect force (N) versus displacement (mm) data for all five cycles. Construct a graph of force (N) versus displacement (mm) separately for all cycles. A representative curve is shown in FIG. 14. From the curve, determine the Wet Maximum Compression Force for each Cycle to the nearest 0.01 N, then multiply by 101.97 and record to the nearest 1 gram-force. Calculate the Wet % Recovery between the First and Second cycle as (TD-E2)/(TD−E1)*100 where TD is the total displacement and E2 is the extension on the second compression curve that exceeds 0.02 N, and record to the nearest 0.01%. In like fashion calculate the Wet % Recovery between the First Cycle and other cycles as (TD−E1)/(TD−E1)*100 and record to the nearest 0.01%. Referring to FIG. 15, calculate the Wet Energy of Compression for Cycle 1 as the area under the compression curve (i.e., area A+B) and record to the nearest 0.1 N-mm. Calculate the Wet Energy Loss from Cycle 1 as the area between the compression and decompression curves (i.e., Area A) and record to the nearest 0.1 N-mm. Calculate the Wet Energy of Recovery for Cycle 1 as the area under the decompression curve (i.e., Area B) and report to the nearest 0.1 N-mm. In like fashion calculate the Wet Energy of Compression (N-mm), Wet Energy Loss (N-mm) and Wet Energy of Recovery (N-mm) for each of the other cycles and record to the nearest 0.1 N-mm.

The overall procedure is now repeated for a total of five replicate wet test specimens, reporting results for each of the five cycles as the arithmetic mean among the five wet replicates for Wet Maximum Compression Force to the nearest 1 gram-force, Wet Energy of Compression to the nearest 0.1 N-mm, Wet Energy Loss to the nearest 0.1 N-mm, and Wet Energy of Recovery to the nearest 0.1 N-mm.

New Artificial Menstrual Fluid (nAMF) Preparation

This formulation of new Artificial Menstrual Fluid (nAMF) is composed of a mixture of defibrinated sheep blood, a phosphate buffered saline solution and a mucous component. The nAMF is prepared such that it has a viscosity between 7.40 to 9.00 centipoise at 23° C.

Viscosity of the nAMF is performed using a low viscosity rotary viscometer (a suitable instrument is the Brookfield DV2T fitted with a Brookfield UL adapter, available from AMETEK Brookfield, Middleboro, MA, or equivalent). The appropriate size spindle for the viscosity range is selected, and the instrument is operated and calibrated as per the manufacturer. Measurements are taken at 23° C.±1° C. and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, sterilized, available from American Laboratories, Inc., Omaha, NE, or equivalent), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or equivalent source.

The phosphate buffered saline solution consists of two individually prepared solutions (Solution A and Solution B). To prepare 1 L of Solution A, add 1.38+0.005 g of sodium phosphate monobasic monohydrate and 8.50+0.005 g of sodium chloride to a 1000 mL volumetric flask and add distilled water to volume. Mix thoroughly. To prepare 1 L of Solution B, add 1.42+0.005 g of sodium phosphate dibasic anhydrous and 8.50+0.005 g of sodium chloride to a 1000 mL volumetric flask and add distilled water to volume. Mix thoroughly. To prepare about 200 mL of phosphate buffered saline solution, add 49.50 g+0.10 g of Solution A and 157.50 g+0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, NC, or equivalent. After the final mixing step, measure and record the viscosity of the mucous component to the nearest 0.01 centipoise at 23° C.±1° C. and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1° C. To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1° C. and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1° C. Any unused portion is discarded after testing is complete.

### Z-Compression Method

The Z-compression method measures the compression behavior along the z-direction of a test specimen, on a Constant Rate of Extension (CRE) universal mechanical test system using a load cell for which the forces measured are within 1% to 99% of the limit of the cell (preferably 100 N). A suitable instrument is the MTS Alliance using TestSuite Software, as available from MTS Systems Corp., Eden Prairie, MN, or equivalent. All testing is performed in a room controlled at 23° C.±3° C. and 50%±2% relative humidity.

The upper and lower fixtures of the test system are circular parallel plate compression platens made of stainless steel. The platen mounted on the moveable CRE fixture has a diameter of 40 mm, and the platen mounted on the stationary CRE fixture has a diameter >40 mm. Both platens have adapters compatible with the mounts of the CRE test machine, capable of securing the platens with their opposing surfaces lying along parallel planes that are orthogonal to the motion of the crossbeam of the CRE test machine.

The absorbent article samples are conditioned at 23° C.±3° C. and 50%±2% relative humidity two hours prior to testing. Remove the test sample from its outer wrapper, then remove the protective cover/release paper from the panty fastening adhesive on the garment facing side of the sample. Lightly apply talc powder to the adhesive to mitigate any tackiness. To obtain a test specimen for measurement, a circular die with a diameter of 40 mm is used. If wings are present on the absorbent article test sample, the test specimen is obtained by centering the circular die over the intersection of the longitudinal midpoint and a lateral line that is centered within the region where the wings are located. If wings are not present, the test specimen is obtained by centering the circular die over the intersection of the longitudinal and lateral midpoints of the absorbent article test sample. In like fashion, five replicate test specimens are prepared from five absorbent article test samples.

Prepare the universal test frame for a compression test to measure force and distance for one cycle of loading (compression) and unloading (recovery) as follows. The crosshead motion is programmed such that the upper platen moves down with respect to the lower platen at a rate of 0.2 mm/s until an endpoint load of 8.66 N (6.9 kPa) is reached, then the crosshead immediately returned to the original gauge (platen separation distance).

Execute the test as follows. Move the platens such that the initial distance between contact surfaces of the platens (gauge) is 25 mm, then zero the crosshead and load cell. Place the test specimen, with the wearer-facing surface upward, onto the bottom platen with the center of the test specimen centered below the upper platen. Manually adjust the position of the upper platen such that its contact surface is about 1 mm above the upper surface of the test specimen. Start the test and continuously collect force (N) and displacement (mm) data at a rate of 100 Hz.

Construct a graph of force (N) versus thickness (mm), across the array of data collected for the entire cycle. To note, at each datapoint, thickness is the original gauge (25 mm) minus the crosshead position (mm). From the resulting force (N) vs thickness (mm) curve, calculate the area under the loading (compression) portion of the curve from the initial thickness to the minimum thickness, and record as energy of compression to the nearest 0.001 N*mm.

In like fashion, the procedure is repeated for all five replicate test specimens. The arithmetic mean across all five replicates is calculated and reported as Energy of Compression to the nearest 0.001 N*mm.

### Peak Load and Elongation Method

The peak load and elongation of a test specimen are measured as the specimen is pulled to failure using a universal constant rate of extension test frame. Measurements are made on test specimens prepared from the machine direction (MD) and the cross direction (CD) of the test material. All measurements are performed in a laboratory maintained at 23° C.±2° C. and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 35 mm wide. The fixtures are installed on the universal test frame and mounted such that they are horizontally and vertically aligned with one another.

Measurements are made on both MD (machine direction) and CD (cross direction) test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained as follows. The MD test specimen is obtained from an area on the test material that is free of any residual of folds or wrinkles. The MD test specimen is cut to a width of 1 inch (25.4 mm) with a length that can accommodate a test span of 51 mm. The long side of the MD test specimen is parallel to the longitudinal axis of the test material as it would appear in an absorbent article. In like fashion, prepare five replicate MD test specimens. The CD test specimen is also cut to a width of 1 inch (25.4 mm) with a length that can accommodate a test span of 51 mm. The long side of the CD test specimen is parallel to the lateral axis of the test material as it would appear in an absorbent article. In like fashion, prepare five replicate CD test specimens.

The universal test frame is prepared for a constant rate of extension tensile test to failure, with slack compensation and gage length adjustment as follows. The initial grip to grip separation distance is set to a nominal gage length (Lₙₒₘᵢₙₐₗ) of 51 mm and then the crosshead is zeroed. The test frame is programmed to move the grips closer together by an intentional slack of 1 mm to ensure no pretension force exists on the test specimen at the onset of the test. (During this motion, the specimen will become slack between the tensile grips.) Next, the grips will move apart at a slack speed of 1 mm/s until the slack preload of 0.10 N is exceeded. At this point, the following are true. 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). The grips will then continue to move apart at a test speed of 254 mm/min until test specimen failure. The grips then return to the nominal gage length.

The test is executed by inserting the MD test specimen into the grips such that the long axis of the specimen is parallel and centered with the motion of the crosshead. The test is started and time, force and displacement data are continuously collected throughout the test at a data acquisition rate of 100 Hz.

A graph of force (N) versus displacement (mm) is constructed. The maximum force value (N) is recorded as MD peak load to the nearest 0.01 N. The crosshead position corresponding to the maximum force value is recorded as Lₚₑₐₖ to the nearest 0.01 mm. The MD percent elongation at the peak load is calculated using the following equation and recorded to the nearest 1 percent.

$$\% Elongation at Peak = ( ( {L}_{peak} - {L}_{o} ) / {L}_{o} ) * 100$$

where Lₒ was previously defined as Lₙₒₘᵢₙₐₗ+L_{slack}. The overall procedure is now repeated until all five MD test specimens have been tested. The arithmetic mean among the five replicate test specimens is calculated for each of the recorded parameters and reported as MD Peak Load to the nearest 0.01 N and MD % Elongation at Peak to the nearest 1 percent.

In like fashion, the entire procedure is repeated for the five CD test specimens, and the arithmetic mean among the five replicate test specimens is calculated and reported as CD Peak Load the nearest 0.01 N and CD % Elongation at Peak to the nearest 1 percent.

### Permeability Measurement Method

This method enables calculation of permeability of a material (in Darcys) via measurement of the downward movement of test fluid through a test specimen along the z-direction (plumb direction), over a range of falling hydrohead indicated by decreasing height of a test fluid in a vessel. The decreasing height of the test fluid inside the vessel, as the fluid drains from the bottom of the vessel through the test specimen, is iteratively measured over time during the procedure. From the collected data together with relevant dimensions of portions of the apparatus through which the fluid moves, the measured wet caliper of the test specimen, and constants associated with gravity and properties of the test fluid chosen, flow rate and permeability may be calculated. All measurements are performed in a laboratory maintained at 23° C.±2° C. and 50%±2% relative humidity and test specimens are conditioned in this environment for at least 2 hours prior to testing.

### Apparatus Components

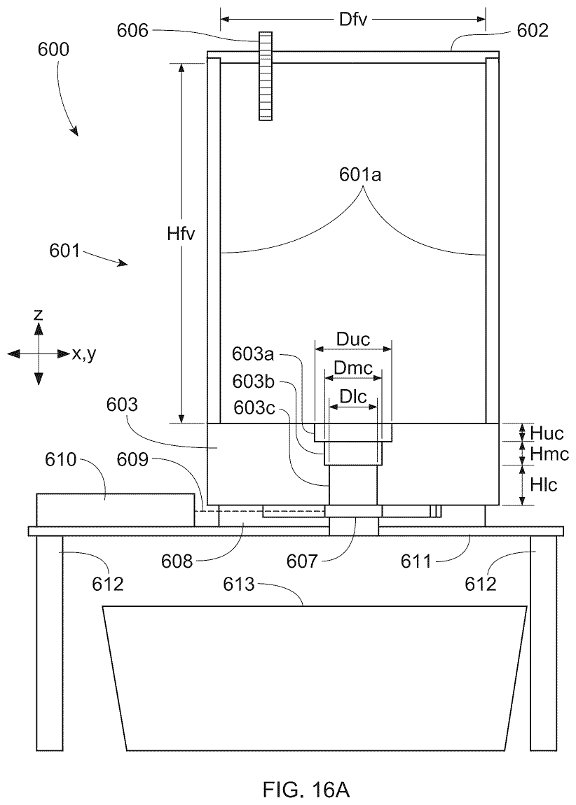

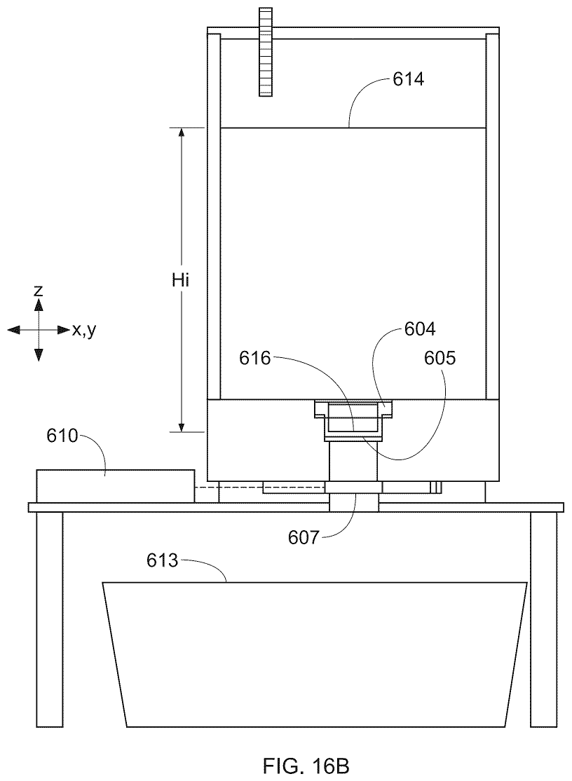

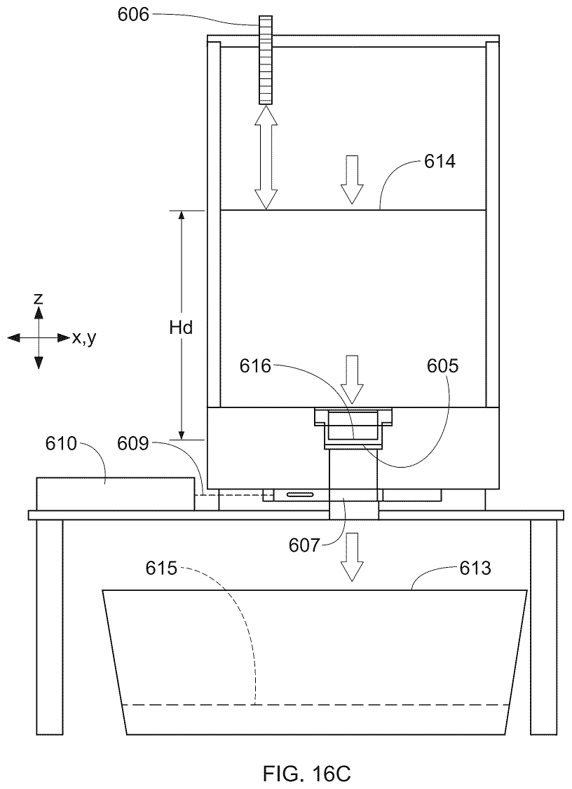

The measurement apparatus 600 and its components are depicted in FIGS. 16a through 18. Referring to FIG. 16a, the apparatus 600 includes a cylindrical fluid vessel 601 including a cylindrical wall 601a with a fitted lid 602 and a base 603 that is sealingly fitted to the bottom of the wall 601a to form fluid vessel 601; a fluid height sensor 606 fitted in and through lid 602; a valve 607 housed in a valve body 608, and a valve actuator 610 mechanically associated with the valve via a linkage 609.

The cylindrical wall has an inside height to the bottom of the lid, Hfv, of 200 mm, an inside diameter of 3⅞ inches (98.425 mm), a wall thickness of ⅜ inch (9.525 mm), and an outer diameter of 4⅝ inches (117.48 mm). The lid 602 is suitably fitted to rest stably on top of the cylindrical wall, but it should not be sealingly fitted thereon; one or more vent holes (not shown) are drilled therethrough to prevent development of negative pressure/vacuum within the fluid vessel as test fluid drains therefrom. The purpose of the lid 602 is to hold and suspend fluid height sensor 606 over the test fluid surface, not to seal the vessel at the top.

Still referring to FIG. 16a, the base 603 has planar, parallel upper and lower surfaces and the upper surface is sealingly affixed to the bottom of the wall 601a. Base 603 is suitably formed or machined to define therewithin a sample chamber having a cylindrical upper chamber portion 603a, a cylindrical middle chamber portion 603b, and a cylindrical lower chamber portion 603c. The three cylindrical chamber portions are coaxial along the vertical/z-direction.

The heights and inner diameters of the three chamber portions are as follows:
Upper chamber portion 603a height Huc: 9.5 mm;
Upper chamber portion 603a inner diameter Duc: 40 mm;
Middle chamber portion 603b height Hmc: 12.5 mm;
Middle chamber portion 603b inner diameter Dmc: 30 mm;
Lower chamber portion 603c height Hlc: 20 mm; and
Lower chamber portion 603a inner diameter Dlc: 26 mm.

Valve body 608 with valve 607 are mounted to the underside of base 603, beneath the lower open end of lower chamber 603c. Valve 607 is configured to be rapidly actuated between fully closed and fully open positions, wherein in the open position, the entirety of lower chamber portion 603c is open to allow fluid to move freely downwardly therefrom without any restriction by valve 607. Valve 607 may be a flat horizontally sliding member, having a circular opening port therethrough, of a diameter of at least 26.0 mm, that is linearly moved to position beneath lower chamber portion 603c upon actuation to the opened position. Alternatively, valve 607 and valve body 608 may have any other suitable configuration adapted to move rapidly between fully closed and fully open positions, wherein when in the fully open position the valve does not present any obstruction to fluid flow downwardly and out from the lower open end of lower chamber portion 603c. Valve 607 and actuator 610 are configured to effect actuation from a fully closed to a fully open position, and vice versa, within no greater than 10 milliseconds for either movement. Actuator 610 may include a solenoid or any other suitable mechanism adapted for this purpose.

Cylindrical wall 601a, lid 602, base 603, and optionally valve body 608 and valve 607, are fabricated of and machined from polished, clear cast acrylic plastic (poly(methyl methacrylate) (PMMA)) stock (known brands include but are not limited to PLEXIGLAS and LUCITE), which may be obtained in various pre-cast tube, rod/bar, disc, sheet and block forms from various suppliers of such materials, such as McMaster-Carr Supply Company (Elmhurst, Illinois). For tube stock used to form wall 601a, tube stock of an inner diameter Dfv varying slightly from that specified herein may be selected, according to availability; in such event, it will be recognized that the corresponding value for the radius r of the fluid vessel, in the equations below, is to be changed to reflect the actual diameter Dfv of the tube stock used.

Fluid height sensor 606 is an ultrasonic height sensor, such as an ML Series part #098-10060, a continuous transmitter through air with an accuracy of about +0.2 mm (TE Connectivity, Schaffhausen, Switzerland and Berwyn, Pennsylvania, USA) or equivalent, interfaced to a computer running software capable of collecting fluid height versus time data throughout the test at a rate of 100 Hz. The fluid height sensor 606 continuously transmits a signal indicating the height of the test fluid within the fluid vessel 601 during the measurement procedure.

The apparatus further includes a support structure, which may include a support platform 611 and height-adjustable legs 612, or any other suitable support structure, configured to stably hold the vessel and valve assembly over a collection vessel 613, with the longitudinal axis of cylindrical wall 601a vertical/plumb and bottom of base 603 level. Where included, a support platform 611 must include an opening or otherwise be configured so as not to obstruct the lower end of lower chamber portion 603c or the fluid exit from valve and valve body 607, 608.

The measurement apparatus further includes a collection vessel 613, of any suitable shape, size and material composition suitable to receive and stably contain the entirety of the volume of test fluid that is used in this method, and fit easily beneath the support structure.

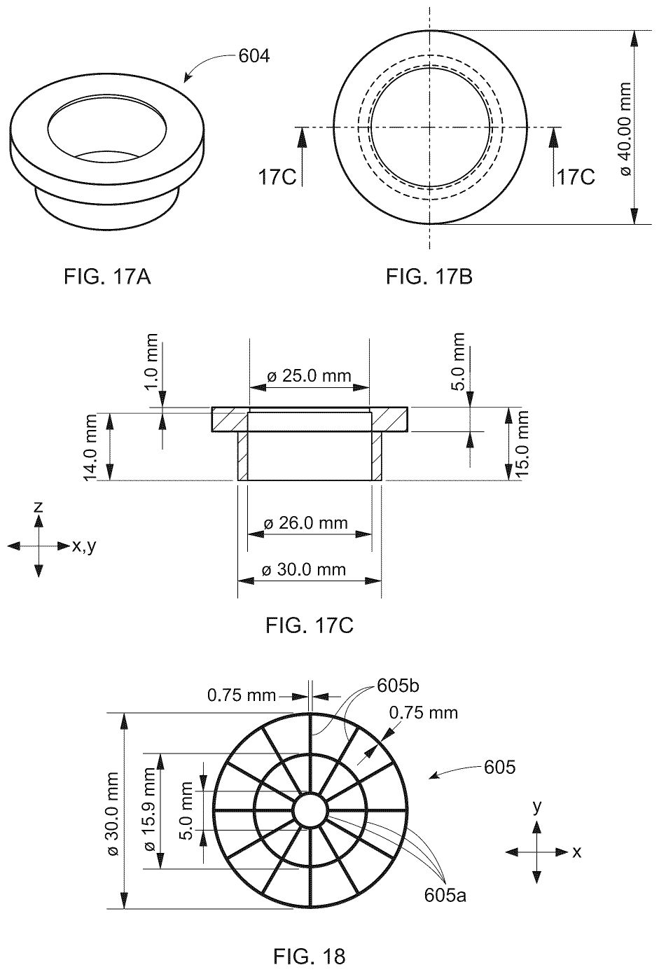

The measurement apparatus further includes a sample weight 604, which is machined of stainless steel to the configuration and dimensions shown in FIGS. 17a-17c. The small radially inwardly-projecting lip at the top portion of sample weight 604 is included for the purpose of providing a feature to grip to facilitate placement and removal of the sample weight 604 into and from the sample chamber.

The measurement apparatus further includes a sample support 605, which has the configuration and dimensions shown in FIG. 18. Sample support 605 has a z-direction caliper of 0.75 mm (which is its height when placed into position within the measurement apparatus in preparation for a measurement procedure). Each of the concentric ring portions 605a and radial spoke portions 605b of sample support 605 shown in FIG. 18 have an x-y plane width of 0.75 mm and a square cross section. Sample support 605 is configured to support a test specimen 616 within middle chamber portion 603b of base 603. Sample support 605 may be cut or machined from any material of suitable strength and corrosion resistance, such as, for example, brass sheet stock.

It will be noted that the outside diameter of sample support 605 and inside diameter of middle chamber portion 603b are both specified above to be 30.0 mm. Sample support 605 is disposed within middle chamber 603b during the measurement procedure. Accordingly, it will be appreciated that either or both of inside diameter of middle chamber portion 603b and outside diameter of sample support 605 may require slight adjustment to provide a small but sufficient clearance to enable sample support 605 to be conveniently inserted into and withdrawn from middle chamber portion 603b.

Similarly, it will be noted that the outside diameter of the lower portion of sample weight 604 and inside diameter of middle chamber portion 603b are both specified above to be 30.0 mm; and the outside diameter of the upper portion of sample weight 604 and inside diameter of upper chamber portion 603a are both specified to be 40.0 mm. The lower portion of sample weight 604 is disposed within middle chamber portion 603b, and the upper portion of sample weight 604 is disposed within upper chamber portion 603a, during the measurement procedure. Accordingly, it will be appreciated that either or both of inside diameter of middle chamber portion 603b and outside diameter of lower portion of sample weight 604, and either or both of inside diameter of upper chamber portion 603a and outside diameter of upper portion sample weight 604, may require slight adjustment to provide a small but sufficient clearance to enable sample weight 605 to be conveniently inserted into and withdrawn from middle chamber portion 603b.

The measurement apparatus further includes a computer (not shown) with suitable software and interfacing equipment configured to communicate with the valve actuator 610 to effect opening and closing of valve 607, and to receive and collect fluid height data from fluid height sensor 606 over time, at a rate of 100 Hz. The person of ordinary skill in the art will have sufficient knowledge and/or resources readily available to obtain components and configure the system including the computer and software to perform the operations described herein.

### Test Fluid Preparation

The test fluid is an aqueous solution of 0.9% w/v saline solution (i.e., 9.0 g of reagent grade NaCl, CAS 7647-14-5, available from any convenient source, diluted to 1 L in deionized water).

Viscosity of the prepared test fluid is performed using a low viscosity rotary viscometer (a suitable instrument is the Cannon LV-2020 Rotary Viscometer with UL adapter, Cannon Instrument Co., State College, Pennsylvania, or equivalent). The appropriate size spindle for the viscosity range is selected, and the instrument is operated and calibrated according to the manufacturer's instructions. Measurements are taken at 23° C.±1° C. and at 30 rpm. Results are reported to the nearest 0.1 centipoise.

### Measurement Procedure

To obtain a test specimen for measurement, lay a single layer of dry subject material out flat on a horizontal work surface, and die-cut a test specimen from it that is circular, with a diameter of 30 mm. Avoid areas of the material having folds, wrinkles or tears when selecting a location for sampling.

If the subject material is a layer component of an absorbent article (e.g., a feminine hygiene pad), for example, a receiving layer or absorbent layer component, obtain a representative sample of the subject material that has not been incorporated into an absorbent article. Alternatively, if only fully manufactured absorbent articles are available as sources of the subject material, from an example thereof, separate the subject layer component from the article without stretching or damaging it. Once the subject layer component has been removed from the article, die-cut out a test specimen as described above. Precondition the test specimen at 23° C.±2° C. and 50%±2% relative humidity for 2 hours prior to testing.

Referring to FIG. 16b, with the fluid valve 607 in closed position, insert the sample support 605 into the middle chamber portion 603b such that it lies horizontal/flat on the lower circumferential lip of middle chamber portion 603b. Using forceps, gently place the test specimen 616 over the sample support 605 so that it lays flat thereon, with no wrinkles. Now gently place the sample weight 604 over/onto the test specimen 616 such that the lower portion of the weight 604 is inserted into middle chamber portion 603b and rests on the test specimen about its circumferential edge, and the upper portion of the weight 604 is nested into the upper chamber portion 603a.

Now slowly add the previously prepared test solution to the fluid vessel 601, until an initial fluid surface 614 height Hi of 150 mm above the upper surface of the test specimen 616 is reached.

Allow the test specimen 616 to equilibrate within the filled sample chamber for about 60 seconds, and ensure there are no bubbles present on the surface of the test fluid or surface of the test specimen. If bubbles are present on the fluid surface, remove or pop them using a clean instrument. If bubbles are present on the upper surface of the test specimen 616, use a clean, round tip lab stirring rod to gently dislodge them, exercising care not to dislodge fibers (if the test specimen is fibrous), or stretch or damage the test specimen.

Secure the fluid height sensor 606 to the lid 602, and then place and fit the lid 602 over cylindrical wall 601a. Adjust the position of the fluid height sensor 606, if necessary, prior to the start of the test so as to prevent it from contacting the starting surface of the test fluid. Initially, the lower tip of the sensor 606 should be about 170 mm from the upper surface of the test specimen 616.

### Position the Collection Vessel 613 Below the Valve 607.

Referring now to FIG. 16c, to start the measurement, simultaneously open the valve 607 and start the acquisition of decreasing fluid height Hd and time data to the nearest 0.01 mm and 0.01 seconds, respectively, with a data acquisition rate of 100 Hz. Test fluid will flow under gravitational pull through the sample chamber and through test specimen 616, sample support 605 and open valve 607, down into collection vessel 613, and test fluid surface 614 will fall while a surface 615 of collected fluid will rise. Height sensor 606 will sense and transmit data concerning the height of test fluid surface 614 at the designated sensing frequency, over time. The measurement is ended and the valve 607 is closed when test fluid has ceased exiting the valve, or after 1,000 seconds have elapsed, whichever occurs first. Remove the lid 602. Lift the sample weight 604 out of the sample chamber, and, using forceps, gently remove the wet test specimen 616 from the sample chamber, and proceed to measure the wet caliper of the test specimen.

The wet caliper of the test specimen 616 is measured promptly after completion of the measurement procedure, using a manually-operated micrometer equipped with a pressure foot capable of exerting a steady pressure of 2.07 kPa+0.07 kPa. The manually-operated micrometer is a dead-weight type instrument with readings accurate to 0.01 mm. A suitable instrument is Mitutoyo Series 543 ID-C Digimatic, available from Avantor/VWR International (Radnor, Pennsylvania) or equivalent. The pressure foot is a flat circular moveable face with a diameter of 19 mm. The test specimen is supported by a horizontal flat reference platform that is larger than and parallel to the surface of the pressure foot. Zero the micrometer against the horizontal flat reference platform. Transfer the wet test specimen 616 to the reference platform of the micrometer such that the specimen 616 is centered and lies horizontally and flat beneath the pressure foot. The pressure foot is lowered by hand with a descent rate of 3+1 mm/s until the full pressure (2.07 kPa) is applied to the test specimen. After 5 seconds elapse, the caliper of the wet test specimen is recorded as specimen caliper, to the nearest 0.01 mm. The test specimen is then discarded.

Remove test fluid inside the fluid vessel 601 and sample chamber, if any remains therein.

The procedure is repeated for a total of three replicate test specimens.

A separate “blank” run measurement is performed by following the procedure described above, but with only the sample support 605 and sample weight 604 present in the sample chamber (i.e., no test specimen is present). Note that the initial test fluid height Hi will be 150 mm above the upper surface of the sample support 605, rather than a surface of a specimen. This blank measurement will enable the permeability of the sample support 605 to be considered, when calculating the permeability of the test specimen.

### Permeability Calculation

Total permeability, kₜₒₜₐₗ, is the permeability of the test specimen plus the sample support, calculated from the time and volume of flow through a fluid height decrease from 150 mm test fluid to 130 mm test fluid. Total permeability is calculated for each replicate test specimen using the following equation, and recorded to the nearest 0.01 E⁻¹⁰ m²:

$$ln ( \frac{Hi}{Hd} ) = \frac{{k}_{total} ρ g}{μ {L}_{total}} * \frac{{R}^{2}}{{r}^{2}} * t$$

thus, solved for kₜₒₜₐₗ:

$${k}_{total} = \frac{[ ( \frac{ln ( \frac{Hi}{Hd} )}{t} ) * \frac{{r}^{2}}{{R}^{2}} * μ * {L}_{total} ]}{ρ g} ;$$

where:
Hi=initial test fluid height (150 mm)
Hd=test fluid height as decreased at time t (for the present calculation, this is 130 mm)
t=time (seconds) elapsed when fluid height has decreased to 130 mm
kₜₒₜₐₗ=combined permeability of the test specimen and the sample support
ρ=density of the test fluid (kg/m³)
g=gravitational constant (9.81 m/s²)
μ=viscosity of the test fluid (assumed to be 0.00109 kg/m-s)
Lₜₒₜₐₗ=combined caliper of the wet test specimen and the sample support (m)
R=the radius of the surface area of the test specimen through which the fluid
flows ((26 mm/2)×(1 m/1,000 mm)=0.013 m)
r=radius of the inside of the fluid vessel ((98.425 mm/2)×(1 m/1,000 mm)=0.049213 m)

The permeability of the sample support 605, kₛₛᵤₚ, is calculated in a similar manner, from the time and volume of flow through a fluid height decrease from 150 mm test fluid to 130 mm test fluid in the “blank” run. The permeability of the sample support 605 alone is described by the following equation, and recorded to the nearest 0.01 E⁻¹⁰ m²:

$$ln ( \frac{Hi}{Hd} ) = \frac{{k}_{ssup} ρ g}{μ {L}_{ssup}} * \frac{{R}^{2}}{{r}^{2}} * t$$

thus, solved for kₛₛᵤₚ:

$${k}_{ssup} = \frac{[ ( \frac{ln ( \frac{Hi}{Hd} )}{t} ) * \frac{{r}^{2}}{{R}^{2}} * μ * {L}_{frit} ]}{ρ g} ;$$

where:
Lₛₛᵤₚ=the caliper of the sample support 605 (0.00075 m)
   The permeability of each replicate test specimen, k_{specimen}, is calculated from the following equation, then multiplied by 1.01324998 E⁺¹² and recorded to the nearest 0.1 Darcy:

$${k}_{specimen} = \frac{{L}_{specimen}}{( \frac{{L}_{total}}{{k}_{total}} ) - ( \frac{{L}_{ssup}}{{k}_{ssup}} )}$$

Now calculate the arithmetic mean of the test specimen permeability, k_{specimen}, across all three replicate test specimens, and report as Through Plane Permeability to the nearest 0.1 Darcy.

### Micro-CT Pore Size Measurement Method

The pore size of a fibrous material composite sample is measured using a micro-CT imaging and analysis method. It is based on analysis of a 3D x-ray sample image obtained on a micro-CT instrument (a suitable instrument is the Scanco μCT 50 available from Scanco Medical AG, Switzerland, or equivalent). The micro-CT instrument is a cone beam micro-tomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and save the raw data. The 3D image is then analyzed using image analysis software (a suitable image analysis software is MATLAB available from The Mathworks, Inc., Natick, MA, or equivalent) to measure the desired properties of regions within the sample.

### Sample Preparation:

The test sample is prepared either from roll stock of the fibrous material composite, or by removing the fibrous material composite material of interest from an absorbent article. A single layer or one or more sublayers of the fibrous material composite test material is placed onto a rigid, horizontal benchtop and a sharp die cutter is used to punch out a circular sample that has a diameter of 7 mm. The test sample is obtained from an area on the test material that is free of folds or wrinkles, and care is used to prevent any contamination or distortion to the test sample during the preparation process. The body facing side of the test sample should be noted and tracked during the analysis portion in order to identify the discrete layers or sublayers present within the test sample. Additional test samples from separate regions within a given test material can be prepared for analysis and comparison. The test samples are conditioned at about 23° C.±2° C. and about 50%±2% relative humidity for 2 hours prior to testing.

### Image Acquisition:

The micro-CT instrument is set up and calibrated according to the manufacturer's specifications. The test sample is placed into the appropriate holder, between two disks of low-density material, which have a diameter of 7 mm. The test sample is scanned under a compressive load by adding a weight to the uppermost low-density disk, with the mass sufficient to apply a pressure of 2 kPa over the 7 mm diameter test sample. Once the compressive load has been applied, the weight is clamped in place to prevent movement during the scan.

The 3D image field of view is approximately 10 mm on each side in the x-y plane with a resolution of approximately 5124 by 5124 pixel, and with a sufficient number of 2 micron thick slices collected to fully include the entire z-direction of the test sample. The reconstructed 3D image contains isotropic voxel of 2 microns. Images are acquired with a source at 45 kVp and 88 μA with no additional low energy filter. The current and voltage setting should be optimized to produce the maximum contrast in the projection data with sufficient x-ray penetration through the test sample, but once optimized, the settings are held constant for all subsequent test sample replicates. A total of 1800 projection images are obtained with an integration time of 750 ms and 6 averages. The projection images are reconstructed into the 3D image and saved in 16-bit format to preserve the full detector output signal for analysis. A file of the resulting data set is of a proprietary format according to the instrument supplier's instruction and is referred to as the ISQ file in the following image visualization and analysis steps.

### Image Visualization and Analysis:

The objective of the image analysis is to measure a 3-dimensional void cell diameter in the first layer (or sublayer) and the second layer (or sublayer) of a fibrous material composite test sample. The ISQ files described above, are read into high end image visualization and analysis platform, for example, Avizo 9.2.0 (FEI, Houston, TX, USA). Upon inspection of obtained visualized 3-dimensional data, 3 different regions in each of the two layers (or sublayers), are analyzed and compared. For each fibrous structure sample there is therefore 6 sub-volumes chosen for measurements of 3-dimensional void size diameter. To make measurements of Porosity and 3D void cell size distribution, the following steps are performed:
1. An automated thresholding algorithm practicing Otsu's method (A Threshold Selection Method from Gray-Level Histograms “, Nobuyuki Otsu, 2EEE Transactions On Systems Man, and Cybernetics, VOL. SMC-9, NO. 1, January 1979) was applied to each of the datasets resulting in a binary image (0-1) representing the fibers (1) and void space (0).
2. A void cell diameter is measured according to the method disclosed in a paper published by Tor Hildebrand (T. Hildebrand and P. Ruegsegger, “A new method for the model-independent assessment of thickness in three-dimensional images. Journal of Microscopy, 185:67-75, 1996). First, the void space is then fitted with spheres of different sizes, where larger spheres cover up smaller spheres using a software working the method disclosed in the paper, for example, IPL software from Scanco Medical, Zurich, Switzerland). This final tessellation of the void space provides a distribution of spheres that completely cover the void space. The volume weighted mean diameter represents the mean void cell diameter. This is implemented through an image analysis platform, for example Matlab R2016B, (Natick, Mass., USA) as module in Avizo 9.2.0.
3. The resulting measurements are brought into Excel 2013. The values of volume weighted mean diameter of the three regions of each layer (or sublayer) are then averaged to produce a single value void cell diameter for that layer (or sublayer). The void cell diameter of the region is a mean pore size of the layer and is reported as Mean Pore Size to the nearest micron.

### % Bonded Volume Measurement Via Micro-CT Method

This micro-CT imaging and analysis method measures the volume percent of fiber bonding present within visually discernible regions of a substrate sample. This method is based on analysis of a 3D x-ray sample image obtained on a micro-CT instrument (a suitable instrument is the Scanco μCT 50 available from Scanco Medical AG, Switzerland, or equivalent). The micro-CT instrument is a cone beam micro-tomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and reconstruction of the raw data into a 3D image. The 3D image is then analyzed using image analysis software (a suitable image analysis software is MATLAB available from The Mathworks, Inc., Natick, MA, or equivalent) to measure the desired properties of regions within the sample.

### Sample Preparation

A test sample is obtained from a single layer of the dry test material in a region of the material that is free from folds, wrinkles, tears, or other defects. A sample having a diameter of 7 mm is cut from the test material using a die or other very sharp blade. The test sample is conditioned in a room maintained at about 23° C.+2° C. and about 50%±2% relative humidity for 2 hours prior to testing.

### Image Acquisition:

The micro-CT instrument is set up and calibrated according to the manufacturer's specifications. The test sample is placed into the appropriate holder and stabilized between low-density material. The sample is positioned such that it is held planarly and aligned with the acquisition planes of the instrument.

The 3D image field of view is approximately 10 mm on each side in the XY-plane with a resolution of approximately 5126 by 5126 pixels, and with a sufficient number of 2 micron thick slices collected to fully include the Z-direction of the test sample. The reconstructed 3D image data set contains isotropic voxels of 2 microns. Images are acquired with the source at 45 kVp and 88 μA with no additional low energy filter. These current, voltage and exposure settings may be optimized to produce the maximum contrast in the projection data with sufficient x-ray penetration through the sample, but once optimized held constant for all substantially similar samples. The projection images are reconstructed into the 3D image and saved in 16-bit format to preserve the full detector output signal for analysis.

### Image Processing:

The 3D image is cropped to a 4 mm by 4 mm square in the XY-plane from the center of the test sample to exclude edge artifacts. The cropped image should be 2000×2000 voxels in the XY-plane and sufficiently thick in the Z direction to enclose the nonwoven. Thus, the cropped sample size will be 2000×2000×ZDEPTH voxels where ZDEPTH is the thickness of the test sample in the Z direction.

A threshold level is chosen that sufficiently separates fiber voxels from all other voxels in the μCT dataset, such as voxels from the low-density stabilizing material. An automated technique such as Otsu's method (implemented as the multithresh function in MATLAB) may be used to find the threshold. Connected components are then used to identify the largest volume in the dataset which represents the test sample. All other volumes will be much smaller and are removed as noise. The “Fiber Mask” resulting from the thresholding will assign fiber voxels a value of one.

Fiber bonding is when two or more fibers are connected by melt. In this method, bonding of fibers can be determined by their morphology. Two morphological operators are used to analyze the “Fiber Mask”. The morphological operators will set the values of fiber voxels based on morphological shapes. Only interior fiber voxels, voxels that are not in contact with empty space, are assigned values but the operators use all fiber voxels to determine the shape. The two morphological operators are the Local Thickness Map (LTM) operator and the Local Bounding Box Map (LBBM) operator.

The LTM operator fits spheres into the interior of fibers. The LTM will assign to a fiber voxel value the diameter of the largest sphere that fits within the fiber and contains that voxel (i.e., LTMDiam). For example, all voxel values within a spherical shaped object would be assigned the diameter of the sphere. This approach is documented in the article by R. P. Dougherty and K-H Kunzelmann, “Computing Local Thickness of 3D Structures with ImageJ,” Microscopy & Microanalysis, August 2007 with the ImageJ plugin code available at https://www.optinav.info/Local_Thickness.htm, both of which are incorporated herein by reference. The ImageJ code was translated into MATLAB for the purposes of this method.

The Local Bounding Box Map (LBBM) operator fits the best “arbitrarily oriented bounding box” to a set of identified points on the surface of the fiber. The LBBM assigns three values which describes the box for each fiber voxel. The LBBM is computed by radiating vectors from a voxel. The vector directions are determined by a geodesic polyhedron which is a polyhedron that can approximate the shape of a sphere when enough triangles are specified. A simple example of a geodesic polyhedron is a Regular Octahedron which is composed of eight equilateral triangles surrounding the voxel. The octahedral used for the LBBM is described as a Class 1, m=4, n=0 octahedral which has 66 vertices and 128 triangles, described and illustrated in detail in the book by Wenninger, M. J. (1979). Spherical Models. Cambridge University Press., incorporated herein by reference. This octahedral was chosen for its large density of nearly uniformly spaced direction vertices and because it has vertices falling on the positive and negative X, Y, Z axes. Each of the 66 vertices are used to define the direction of vectors radiating from the interior fiber voxel to the first surface voxel encountered. This generates 66 points in space. If the vector does not reach a surface point within a maximum distance of 25 voxels, then the voxel point at the maximum distance is reported. Note that the 66 points may not be distinct if the interior voxel falls close to the fiber surface.

The 66 points generated by the LBBM operator are then used to find an Arbitrarily Oriented Minimum Bounding Box (AOMBB), which is generally described in the article by O'Rourke, Joseph. “Finding Minimal Enclosing Boxes.” International Journal of Computer and Information Sciences, vol. 14, no. 3, pp. 183-199, June 1985., incorporated herein by reference. This is the smallest box located anywhere in space, at any angle that will enclose all 66 points. The size of the box is described by its length, width, and height values. For AOMBB, the largest of these three values will be reported as the maximum distance (MaxBB), the smallest of these three values as the minimum distance (MinBB), and the remaining value as the middle-distance measure (MidBB). These three values (MaxBB, MidBB, MinBB) are determined and assigned independently to each interior fiber voxel. The AOMBB algorithm used to compute these three values came from the MATLAB file exchange [Johannes Korsawe (2022). Minimal Bounding Box (https://www.mathworks.com/matlabcentral/fileexchange/18264-minimal-bounding-box), MATLAB Central File Exchange. Retrieved Feb. 22, 2022], incorporated herein by reference. Note that voxels that fall within the same bounding box will be assigned the same values. This is like the LTM where voxels within the same sphere will have the same value.

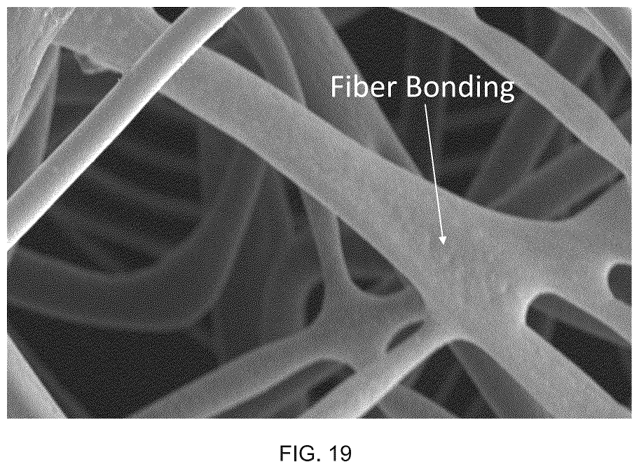

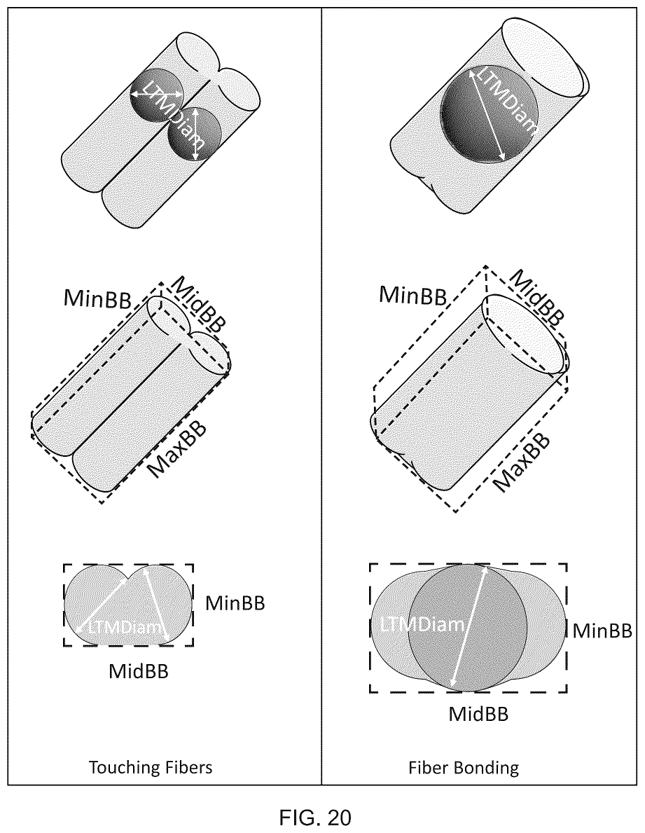

Fiber bonding occurs when two fibers are in contact, and their outer layers have melted together. An example of fiber bonding is shown in the SEM image of FIG. 19. Two fibers that have not formed a bond but are simply touching will maintain their nearly uniform diameter. FIG. 20 shows the expected morphological measures when fibers are merely touching (left column) versus when two fibers bond (right column). Referring to the left column of FIG. 20, two touching fibers will share voxels where they touch but maintain a cylindrical shape. The LTMDiam which assigns voxel values based on the largest sphere will record the diameters of the fibers. With regards to the LBBM, the voxel's MaxBB length will usually follow the center line of the fiber. Therefore, the voxel's MinBB measure will be similar to the fiber diameter while the MidBB value should be twice the LTMDiam value (0.5*MidBB˜=LTMDiam). The right column of FIG. 20 shows two bonded fibers with melt between them, hence the LTMDiam includes the melt material and is greater than the fiber diameter. Therefore, 0.6*MidBB<LTMDiam, indicates the presence of melt and would be counted as bonding. The 0.6 multiplier is used to account for noise.

For bicomponent (which may be referred to herein as bico) fibers, the melt is generated from the outer sheath of the fiber. Therefore, the MidBB need not be twice the MinBB as in the case of no bonding. However, the MidBB should be greater than the MinBB indicating a more elliptical shape that includes the core of two fibers. MinBB equal to MidBB would appear as a single fiber. Therefore, MidBB>1.3*MinBB indicates an elliptical shape which likely contains two fibers. An interior fiber voxel will be labeled part of a fiber bonding when its morphological values satisfy the equation:

$$IsBonding = ( 0.6 * MidBB < LTMDiam ) \& ( MidBB > 1.3 * Min BB )$$

The first part of the equation indicates material melt which is logically “AND” with the second part of the equation that indicates greater width than one fiber.

### Measurement of Bond Volume:

The 2000×2000×ZDEPTH voxel dataset are divided into sixteen 500×500×ZDEPTH subsamples so that less computer processing time is required to process the morphological operations. The four corner subsamples are excluded from the analysis due to their proximity to edge artifacts. Thus, a total of nine subsamples are processed. Each interior pixel of the fiber mask is labeled using the IsBonding equation previously described. Noise in the dataset is cleaned up by removing the IsBonding label from connected clusters of IsBonding voxels that number less than 100. As previously discussed, only interior voxels are assigned a label. The interior voxels are morphologically dilated by one voxel in all directions to account for surface voxels. Dilated voxels that fall outside of the original fiber mask are removed. The volume percentage is calculated by dividing the number of IsBonding labeled voxels by the total number of fiber voxels in the subsample and recorded to the nearest 0.1 percent. The arithmetic mean of the volume percentage values across all nine subsamples is calculated and reported as % Bonded Volume to the nearest 0.1 percent.

### Bending Length

For the Bending Length testing; compendial method WSP 90.5 (05) is followed.

### Combinations

A1. A fluid management layer comprising: a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers, wherein the fluid management layer has a caliper factor of from about 0.26 to about 0.40, and wherein the cellulosic fibers and the bonding fibers have a decitex no greater than about 2.

A2. The fluid management layer according to paragraph A1, further comprises integrated stitches at a stitch density of between 90 and 220 punches per square centimeter.

A3. The fluid management layer according to paragraph A2, wherein the stitch direction is selected from the top, bottom and combinations thereof.

A4. The fluid management layer according to any one of the preceding paragraphs, wherein the fiber management layer has an MD: CD Peak Load ratio from about 0.5 to about 1.75.

A5. The fluid management layer according to any one of the preceding paragraphs, wherein the divider fibers having a decitex of from about 0.5 to about 2.

A6. The fluid management layer according to paragraph A5, wherein the divider fibers comprise at least one of polypropylene, polyethylene terephthalate, bicomponent polyethylene, bicomponent polypropylene, and bicomponent polyethylene terephthalate.

A7. The fluid management layer according to paragraph A6, wherein the divider fibers are non-cylindrical polypropylene fibers.

A8. The fluid management layer according to any one of the preceding paragraphs, wherein the cellulosic fibers comprise at least one of cotton, rayon, viscose, lyocell, natural cellulose, and regenerated cellulose.

A9. The fluid management system according to paragraph A8, wherein the cellulosic fibers are viscose.

A10. The fluid management layer according to any one of the preceding paragraphs, wherein the bonding fibers comprise at least one of bicomponent polyethylene terephthalate/polyethylene, combinations of polyethylene, polypropylene, polyethylene terephthalate, co-polyethylene terephthalate.

A11. The fluid management layer according to paragraph A10, wherein the bonding fibers are polyethylene terephthalate/polyethylene wherein the core is polyethylene terephthalate and the sheath is polyethylene.

A12. The fluid management layer according to any one of the preceding paragraphs, wherein the bonding fibers comprise bicomponent fibers.

A13. The fluid management layer according to paragraph A12, wherein the bonding fibers further comprise non-cylindrical polymeric fibers.

A14. The fluid management according to any one of the preceding paragraphs, wherein cellulosic fibers have a decitex of from about 0.5 to about 1.7.

A15. The fluid management layer according to any one of the preceding paragraphs, wherein the bonding fibers have a decitex of from about 1 to about 2.

A16. The fluid management layer according to any one of the preceding paragraphs, wherein the fluid management layer has a MD peak load of from about 4 to about 85 Newtons.

A17. The fluid management layer according to any one of the preceding paragraphs, wherein the fluid management layer has a CD peak load of from about 4 to about 130 Newtons.

A18. The fluid management layer according to any one of the preceding paragraphs, wherein the average pore size is from about 40 to about 150 μm.

A19. The fluid management layer according to paragraph A18, wherein the average pore size is from about 50 to about 120 μm.

A20. The fluid management layer according to any one of the preceding paragraphs, wherein the fibers are from about 10 to about 120 mm in length.

A21. The fluid management layer according to paragraph A20, wherein the fibers are from about 24 to about 95 mm in length.

A22. The fluid management layer according to paragraph A21, wherein the fibers are from about 36 to about 75 mm in length.

A23. The fluid management layer according to any one of the preceding paragraphs, wherein the fibers have a fibers length selected from a same length, a different length, or combinations thereof.

B1. A disposable absorbent article comprising a topsheet, a backsheet, an absorbent core disposed between the topsheet and the backsheet, and a fluid management disposed between the topsheet and the absorbent core wherein the fluid management layer comprises a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers, wherein the fluid management layer has a caliper factor of from about 0.26 to about 0.40, and wherein the cellulosic fibers and the polymeric fibers have a decitex less than about 2.

B2. The absorbent article according to paragraph B1, wherein the absorbent article has a Z-compression compression energy of from about 2.6 N·mm to about 4.0 N·mm, a 3 point MD bend dry bending stiffness of from about 15 N·mm\hat{ }2 and to about 40 55 N·mm\hat{ }2, and a wet bunch compression % recovery of greater than about 40%.

B3. The absorbent article according to paragraph B2, wherein the topsheet comprises an anti-stick agent on the wearer facing side of the topsheet.

B4. The absorbent article according to paragraph B3, wherein the anti-stick agent comprises a propylene glycol material.

B5. The absorbent article according to paragraph B4, wherein the propylene glycol material is polypropylene glycol.

B6. The absorbent article according to any one of the preceding paragraphs, wherein the divider fibers are selected from polypropylene, polyethylene terephthalate, bicomponent polyethylene, bicomponent polypropylene, bicomponent polyethylene terephthalate and combinations thereof.

C1. A fluid management layer comprising: a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers, wherein the cellulosic fibers, the bonding fibers, and the divider fibers have a decitex of less than about 2.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fluid management layer comprising:
a nonwoven having a basis weight of from about 40 gsm to about 85 gsm,
from about 15 to about 35 weight percent of cellulosic fibers,
from about 20 to about 40 weight percent of bonding fibers, and
from about 40 to about 60 weight percent of divider fibers,
wherein the fluid management layer has a caliper factor of from about 0.26 to about 0.40, and wherein the cellulosic fibers and the bonding fibers have a decitex no greater than about 2.

2. The fluid management layer of claim 1, further comprises integrated stitches at a stitch density of between 90 and 220 punches per square centimeter.

3. The fluid management layer of claim 1, wherein the fiber management layer has an MD: CD Peak Load ratio from about 0.5 to about 1.75.

4. The fluid management layer of claim 1, wherein the divider fibers having a decitex of from about 0.5 to about 2.

5. The fluid management layer of claim 4, wherein the divider fibers comprise at least one of polypropylene, polyethylene terephthalate, bicomponent polyethylene, bicomponent polypropylene, and bicomponent polyethylene terephthalate.

6. The fluid management layer of claim 5, wherein the divider fibers are non-cylindrical polypropylene fibers.

7. The fluid management layer of claim 1, wherein the cellulosic fibers comprise at least one of cotton, rayon, viscose, lyocell, natural cellulose, and regenerated cellulose.

8. The fluid management system of claim 7, wherein the cellulosic fibers are viscose.

9. The fluid management layer of claim 1, wherein the bonding fibers comprise at least one of bicomponent polyethylene terephthalate/polyethylene, combinations of polyethylene, polypropylene, polyethylene terephthalate, co-polyethylene terephthalate.

10. The fluid management layer of claim 9, wherein the bonding fibers are polyethylene terephthalate/polyethylene wherein the core is polyethylene terephthalate and the sheath is polyethylene.

11. The fluid management layer of claim 1, wherein the bonding fibers comprise bicomponent fibers.

12. The fluid management layer of claim 11, wherein the bonding fibers further comprise non-cylindrical polymeric fibers.

13. The fluid management layer of claim 1, wherein cellulosic fibers have a decitex of from about 0.5 to about 1.7.

14. The fluid management layer of claim 1, wherein the bonding fibers have a decitex of from about 1 to about 2.

15. The fluid management layer of claim 1, wherein the fluid management layer has a MD peak load of from about 4 to about 85 Newtons.

16. The fluid management layer of claim 1, wherein the fluid management layer has a CD peak load of from about 4 to about 130 Newtons.

17. The fluid management layer of claim 1, wherein the average pore size is from about 40 μm to about 150 μm.

18. The fluid management layer of claim 17, wherein the average pore size is from about from about 50 μm to about 120 μm.

19. The fluid management layer of claim 1, wherein the fibers length is selected from a same length, a different length, or combinations thereof.

20. A disposable absorbent article comprising a topsheet, a backsheet, an absorbent core disposed between the topsheet and the backsheet, and a fluid management disposed between the topsheet and the absorbent core wherein the fluid management layer comprises a nonwoven having a basis weight of from about 40 gsm to about 85 gsm, from about 15 to about 35 weight percent of cellulosic fibers, from about 20 to about 40 weight percent of bonding fibers, and from about 40 to about 60 weight percent of divider fibers, wherein the fluid management layer has a caliper factor of from about 0.12 to about 0.25, and wherein the cellulosic fibers and the polymeric fibers have a decitex less than about 2.

21. The absorbent article of claim 20, wherein the absorbent article has a Z-compression compression energy of from about 2.6 N·mm to about 4.0 N·mm, a 3 point MD bend dry bending stiffness of from about 15 N·mm\hat{ }2 to about 55 N·mm\hat{ }2, and a wet bunch compression % recovery of greater than about 40%.

22. The absorbent article of claim 21, wherein the wet bunch compression % recovery is less than about 70%.

23. A fluid management layer comprising:
a nonwoven having a basis weight of from about 40 gsm to about 85 gsm,
from about 15 to about 35 weight percent of cellulosic fibers,
from about 20 to about 40 weight percent of bonding fibers, and
from about 40 to about 60 weight percent of divider fibers,
wherein the cellulosic fibers, the bonding fibers, and the divider fibers have a decitex of less than about 2.
